SEQUENCE LISTING (41 sequences)

SEQ ID NO: 1 - protein, 5 aa, synthetic construct
ARLRK

SEQ ID NO: 2 - DNA, 22 bp, synthetic construct
  source 1..22: mol_type = other DNA
caccattggcaatgagcggttc

SEQ ID NO: 3 - DNA, 22 bp, synthetic construct
  source 1..22: mol_type = other DNA
aggtctttgcggatgtccacgt

SEQ ID NO: 4 - DNA, 22 bp, synthetic construct
  source 1..22: mol_type = other DNA
gtgcctatgtctcagcctcttc

SEQ ID NO: 5 - DNA, 22 bp, synthetic construct
  source 1..22: mol_type = other DNA
gccatagaactgatgagaggga

SEQ ID NO: 6 - DNA, 22 bp, synthetic construct
  source 1..22: mol_type = other DNA
ccacagaccttccaggagaatg

SEQ ID NO: 7 - DNA, 23 bp, synthetic construct
  source 1..23: mol_type = other DNA
gtgcagttcagtgatcgtacagg

SEQ ID NO: 8 - DNA, 23 bp, synthetic construct
  source 1..23: mol_type = other DNA
agaatcaccagcagcaagtgtcc

SEQ ID NO: 9 - DNA, 22 bp, synthetic construct
  source 1..22: mol_type = other DNA
tcctgaacccacttctgcttgg

SEQ ID NO: 10 - DNA, 22 bp, synthetic construct
  source 1..22: mol_type = other DNA
cctgctgctttgcctacattgc

SEQ ID NO: 11 - DNA, 22 bp, synthetic construct
  source 1..22: mol_type = other DNA
acacacttggcggttctttcgg

SEQ ID NO: 12 - DNA, 24 bp, synthetic construct
  source 1..24: mol_type = other DNA
ccatcagcttgtctgtttcattcc

SEQ ID NO: 13 - DNA, 23 bp, synthetic construct
  source 1..23: mol_type = other DNA
gctgtaatccaaggaatgtggtc

SEQ ID NO: 14 - DNA, 23 bp, synthetic construct
  source 1..23: mol_type = other DNA
cgagcatcattctttcaccagct

SEQ ID NO: 15 - DNA, 22 bp, synthetic construct
  source 1..22: mol_type = other DNA
atgagaggcagcaagatggacc

SEQ ID NO: 16 - DNA, 22 bp, synthetic construct
  source 1..22: mol_type = other DNA
gaaaggctacgtgactcctgtg

SEQ ID NO: 17 - DNA, 22 bp, synthetic construct
  source 1..22: mol_type = other DNA
gtctaccagattctgctcactc

SEQ ID NO: 18 - DNA, 22 bp, synthetic construct
  source 1..22: mol_type = other DNA
tggatcaccactggcatctctg

SEQ ID NO: 19 - DNA, 22 bp, synthetic construct
  source 1..22: mol_type = other DNA
gctccaggttgtgaagcatcac

SEQ ID NO: 20 - DNA, 22 bp, synthetic construct
  source 1..22: mol_type = other DNA
gcttcgatgcacgggaacaatg

SEQ ID NO: 21 - DNA, 22 bp, synthetic construct
  source 1..22: mol_type = other DNA
cattggtgtggatgcagatccg

SEQ ID NO: 22 - DNA, 23 bp, synthetic construct
  source 1..23: mol_type = other DNA
catcactgccacccagaagactg

SEQ ID NO: 23 - DNA, 23 bp, synthetic construct
  source 1..23: mol_type = other DNA
atgccagtgagcttcccgttcag

SEQ ID NO: 24 - DNA, 22 bp, synthetic construct
  source 1..22: mol_type = other DNA
ggtgcctatgtctcagcctctt

SEQ ID NO: 25 - DNA, 23 bp, synthetic construct
  source 1..23: mol_type = other DNA
gccatagaactgatgagagggag

SEQ ID NO: 26 - DNA, 22 bp, synthetic construct
  source 1..22: mol_type = other DNA
tggaccttccaggatgaggaca

SEQ ID NO: 27 - DNA, 22 bp, synthetic construct
  source 1..22: mol_type = other DNA
gttcatctcggagcctgtagtg

SEQ ID NO: 28 - DNA, 22 bp, synthetic construct
  source 1..22: mol_type = other DNA
gctacaagaggatcaccagcag

SEQ ID NO: 29 - DNA, 22 bp, synthetic construct
  source 1..22: mol_type = other DNA
gtctggacccattccttcttgg

SEQ ID NO: 30 - DNA, 22 bp, synthetic construct
  source 1..22: mol_type = other DNA
cctgctgctttgcctacctctc

SEQ ID NO: 31 - DNA, 22 bp, synthetic construct
  source 1..22: mol_type = other DNA
acacacttggcggttccttcga

SEQ ID NO: 32 - DNA, 24 bp, synthetic construct
  source 1..24: mol_type = other DNA
acgtattggaaggagattacagct

SEQ ID NO: 33 - DNA, 22 bp, synthetic construct
  source 1..22: mol_type = other DNA
tctgtcagcgttactatcccgc

SEQ ID NO: 34 - DNA, 22 bp, synthetic construct
  source 1..22: mol_type = other DNA
ggcggtggaatacaatgtgtcc

SEQ ID NO: 35 - DNA, 22 bp, synthetic construct
  source 1..22: mol_type = other DNA
agcaggtcaaggtgaacagctg

SEQ ID NO: 36 - DNA, 22 bp, synthetic construct
  source 1..22: mol_type = other DNA
ggaaaatggaggtctggactcg

SEQ ID NO: 37 - DNA, 23 bp, synthetic construct
  source 1..23: mol_type = other DNA
gtgtcattagccacagcgaactc

SEQ ID NO: 38 - DNA, 22 bp, synthetic construct
  source 1..22: mol_type = other DNA
gcatagaggcagacgcttccta

SEQ ID NO: 39 - DNA, 22 bp, synthetic construct
  source 1..22: mol_type = other DNA
ccactgcttctttcagggcatc

SEQ ID NO: 40 - DNA, 22 bp, synthetic construct
  source 1..22: mol_type = other DNA
agtcaacgtggaggtgtctgct

SEQ ID NO: 41 - DNA, 22 bp, synthetic construct
  source 1..22: mol_type = other DNA
gtagactccacctgaaaccagg

(19) United States
(12) Patent Application Publication
(10) Pub. No.: US 2026/0115311 A1
(43) Pub. Date: Apr. 30, 2026

(54) INFLAMMATORY CONDITION-SPECIFIC DRUG-RELEASING HYDROGEL FOR OCULAR INJECTION

(71) Applicants: UNIVERSITY-INDUSTRY COOPERATION GROUP OF KYUNG HEE UNIVERSITY, Yongin-si (KR); KOREA INSTITUTE OF SCIENCE AND TECHNOLOGY, Seoul (KR)

(72) Inventors: Seung Ja OH, Seoul (KR); Maesoon IM, Seoul (KR); Hyeonhee ROH, Seoul (KR); Hyerim KIM, Seoul (KR)

(73) Assignees: UNIVERSITY-INDUSTRY COOPERATION GROUP OF KYUNG HEE UNIVERSITY, Yongin-si (KR); KOREA INSTITUTE OF SCIENCE AND TECHNOLOGY, Seoul (KR)

(21) Appl. No.: 19/235,813

(22) Filed: Jun. 12, 2025

(30) Foreign Application Priority Data
Oct. 25, 2024 (KR) 10-2024-0147815

(51) Int. Cl. A61K 47/69; A61K 9/00; A61K 47/06; A61K 47/36; A61K 47/65

(52) U.S. Cl. CPC A61K 47/6903; A61K 9/0019; A61K 9/0048; A61K 47/06; A61K 47/36; A61K 47/65

(57) ABSTRACT

The disclosure relates to inflammatory condition-specific drug-releasing hydrogels for ocular injection and uses thereof. According to a hydrogel according to an aspect, the release of a drug may be controlled selectively for an inflammatory condition, and direct intraocular injection is possible.

### CROSS-REFERENCE TO RELATED APPLICATION

This application is based on and claims priority under 35 U.S.C. § 119 to Korean Patent Application No. 10-2024-0147815, filed on Oct. 25, 2024, in the Korean Intellectual Property Office, the disclosure of which is incorporated by reference herein in its entirety.

### INCORPORATION-BY-REFERENCE OF MATERIAL SUBMITTED BY U.S.P.T.O. EFS-WEB

This application contains a Sequence Listing, which is being submitted in computer readable form via the United States Patent and Trademark Office Patent Center and which is hereby incorporated by reference in its entirety for all purposes. The XML file submitted herewith, which is named as “NewApp_1370550001_SequenceListings” and is created on Jun. 11, 2025, contains a 36.2 KB file.

### BACKGROUND

### 1. Field

The disclosure relates to an inflammatory condition-specific drug-releasing hydrogel for ocular injection and uses thereof, and more particularly, to a hydrogel capable of controlling the release of a drug in an inflammatory condition-specific manner, a composition including the hydrogel for drug delivery, anti-inflammatory use, or ocular disease treatment, and a method of preparing the hydrogel.

### 2. Description of the Related Art

Retinitis pigmentosa (RP) is an outer retinal degenerative disease that may lead to photoreceptor cell apoptosis and severe vision loss. Effective control of intraretinal inflammation may slow disease progression, but efficient anti-inflammatory treatment strategies are still lacking.

Effective resolution of inflammation contributes to preventing disease progression and promoting healing. In the past, most studies focused on immediate reduction of inflammation, but recent studies have reported that appropriate control of inflammation severity may reduce drug side effects and enhance therapeutic effects, making timely control of inflammation more important.

An important regulation in resolving the inflammatory environment is the regulation of macrophage plasticity, and the conversion of inflammatory macrophages, M1, into anti-inflammatory macrophages, M2, is a key factor in controlling the transition to the resolution phase of the inflammatory process. Epigenetic regulation may be an effective strategy to alter macrophage phenotype due to its powerful ability to modulate macrophage plasticity with target specificity and reversibility. Therefore, alleviating inflammatory responses through epigenetic regulation of macrophages may be a novel therapeutic strategy in clinical applications. However, since drugs for epigenetic regulation have very short half-lives and the epigenome is sensitively regulated depending on the environment, drugs for epigenetic regulation must be delivered to the epigenome in a timely manner to maximize the desired effect. Therefore, in order to control inflammation through effective epigenetic regulation of macrophages, a drug delivery system that may deliver drugs to macrophages in a timely and inflammatory condition-specific manner is required.

Hydrogels have high biocompatibility due to their high moisture content, porous structure, and similarity to the extracellular matrix. Due to these characteristics, they are being widely studied in biomedical fields such as drug delivery systems and tissue engineering.

Meanwhile, research on drug delivery systems that may release drugs in an inflammatory condition-specific manner and enable ocular injection remains insufficient.

### SUMMARY

Provided is a hydrogel formed by cross-linking hyaluronic acid with a crosslinker, wherein a drug is encapsulated within the hydrogel, and the crosslinker includes a peptide that is degraded by cathepsins.

Provided is a drug delivery composition including the hydrogel.

Provided is an anti-inflammatory skin external application composition including the hydrogel.

Provided is a pharmaceutical composition for treating an ocular disease, including the hydrogel.

Provided is a method of treating an ocular disease, including administering to a subject a therapeutically effective amount of the hydrogel, wherein the hydrogel is a hydrogel formed by cross-linking hyaluronic acid with a crosslinker, a drug is encapsulated within the hydrogel, and the crosslinker includes a peptide that is degradable by cathepsins.

Provided is a method of preparing a hydrogel, including (1) a first step of preparing dibenzylcyclooctyne (DBCO)-conjugated hyaluronic acid (DBCO-HA), (2) a second step of mixing the DBCO-HA with a solvent, (3) a third step of mixing a drug, and (4) a fourth step of adding a crosslinker, wherein the crosslinker includes a peptide degradable by cathepsins.

Additional aspects will be set forth in part in the description which follows and, in part, will be apparent from the description, or may be learned by practice of the presented embodiments of the disclosure.

According to an aspect of the disclosure, provided is a hydrogel formed by cross-linking hyaluronic acid with a crosslinker, wherein a drug is encapsulated within the hydrogel, and the crosslinker includes a peptide that is degraded by cathepsins.

The hydrogel may be formed by cross-linking the hyaluronic acid with a peptide that is degraded or cleaved by the cathepsin, and a drug may be encapsulated within the hydrogel.

That is, the hydrogel may be a hydrogel formed by cross-linking hyaluronic acid as a crosslinker, wherein a drug is encapsulated within the hydrogel, and the crosslinker includes a peptide that is degraded or cleaved by cathepsin.

The hyaluronic acid may be DBCO-conjugated hyaluronic acid (DBCO-HA) to which DBCO is conjugated.

The DBCO-HA may be a product in which the carboxyl group of hyaluronic acid and DBCO are linked by copper-free click chemistry.

Copper-free click chemistry has the advantage of being highly biocompatible as it does not require a copper catalyst, and since it does not use light (especially ultraviolet rays, UV), there is no phototoxicity or copper ion toxicity to cells or living tissues. On the other hand, UV cross-linking or photo-initiated click chemistry uses light of specific wavelengths, such as ultraviolet rays or visible light, etc., and during this process, high-energy UV rays may cause DNA damage, cell damage, and phototoxicity, which may be problematic, especially in in vivo studies or cell culture experiments.

In addition, copper-free click chemistry may cause reactions without light or special photoinitiators, and the experimental environment is simpler and no special equipment (UV lamp or light source of a specific wavelength) is required, therefore the experimental process is simple and costs may be reduced.

Additionally, copper-free click chemistry has a very fast reaction rate, therefore the reaction may be completed in a relatively short period of time.

Furthermore, copper-free click chemistry has high selectivity as it reacts only between very specific reactants such as DBCO and azide.

According to an aspect of the disclosure, the peptide that is degraded or cleaved by the cathepsin may include an amino acid sequence of SEQ ID NO: 1.

The hydrogel may have a network structure in which the hyaluronic acid is cross-linked with a peptide that is degraded or cleaved by the cathepsin, and the drug may be encapsulated within the network structure.

According to an aspect of the disclosure, when the crosslinker is degraded or cleaved by cathepsin, the drug may be released outside the hydrogel. That is, when the peptide that functions as a crosslinker is degraded or cleaved by the cathepsin, the cross-linking of the hydrogel may be damaged or destroyed, thereby damaging or destroying the network structure, and as a result, the drug encapsulated inside the hydrogel may be released to the outside.

Therefore, the hydrogel may selectively release the drug in the presence of cathepsin, upon contact with cathepsin, or when exposed to cathepsin. The cathepsin is an enzyme expressed or secreted from macrophages during an inflammatory response. Ultimately, the hydrogel may selectively release the drug at an inflammatory lesion or when exposed to an inflammatory environment or in contact with an inflammatory environment. The inflammation may exist within the body, and specifically may exist within the skin or on the skin surface.

According to an aspect of the disclosure, the hydrogel has different drug release levels depending on the degree of inflammation progression, so that drug release may be controlled depending on the state of inflammation progression. Specifically, according to an aspect of the disclosure, the hydrogel delays the release of the drug and protects the drug from the external environment in a normal environment or an environment with a low concentration of activated cathepsin, while selectively releasing the drug only when exposed to an inflammatory environment, and in particular, may continuously release the drug from the stage with a high concentration of activated cathepsin, in other words, the acute inflammatory phase, through the subsequent proliferative phase.

According to an aspect of the disclosure, the hydrogel may control the release of a drug with maintained activity at the most appropriate time to increase therapeutic efficiency throughout the entire course of an inflammatory response, thereby maximizing the effect of the drug (for example, a drug for epigenetic regulation of macrophages) and exhibiting remarkably excellent anti-inflammatory, cell regeneration (for example, promoting migration and proliferation of keratinocytes), and wound healing effects.

The peptide that is degraded or cleaved by the cathepsin may be degraded or cleaved by the cathepsin by hydrolysis by the cathepsin, thereby destroying the peptide bond.

The drug may be a drug that exhibits an anti-inflammatory effect or helps in wound healing, and may be one commonly used in the art. For example, the drug may be, but is not limited to, an antibiotic, a wound healing promoter, a disinfectant, a local anesthetic, an anti-inflammatory agent, etc.

According to an aspect of the disclosure, the drug may be a drug that exhibits an anti-inflammatory effect through epigenetic regulation of macrophages.

According to an aspect of the disclosure, the drug may be, but is not limited to, an enhancer of zeste homolog 2 (EZH2) inhibitor.

The average molecular weight of the drug encapsulated in the hydrogel may be about 600 to about 10,000 g·mol⁻¹. Specifically, the average molecular weight of the drug may be about 600 to about 8,000 g·mol⁻¹ or about 600 to about 5,000 g·mol⁻¹.

According to an aspect of the disclosure, when the average molecular weight of the drug is less than about 600 g·mol⁻¹, the drug-encapsulated hydrogel may release the drug not only in an inflammatory environment where cathepsin exists but also in a normal environment, so that the specificity for an inflammatory condition in drug release may decrease, and when the average molecular weight of the drug is more than about 10,000 g·mol⁻¹, the drug-encapsulated hydrogel may have a decreased drug encapsulation efficiency or a decreased release amount of the encapsulated drug, so that the anti-inflammatory effect may decrease.

The hydrogel may include the crosslinker in a molar concentration of about 3 mM or more. Specifically, the hydrogel may include the crosslinker in a molar concentration of about 3 to about 50 mM, about 3 to about 40 mM, about 3 to about 30 mM, about 3 to about 20 mM, about 3 to about 10 mM, or about 3 to about 5 mM.

According to an aspect of the disclosure, when the molar concentration of the crosslinker included in the hydrogel is less than about 3 mM, the mechanical properties of the hydrogel, such as network density and hardness, etc., decrease, so that the hydrogel may release drugs not only in an inflammatory environment in which cathepsin exists but also in a normal environment, and thus the specificity for an inflammatory condition in drug release may decrease, and when the molar concentration of the crosslinker included in the hydrogel exceeds about 50 mM, the hydrogel may have a decreased drug encapsulation efficiency or a decreased release amount of the encapsulated drug, so that the anti-inflammatory effect may decrease.

The Young's modulus value of the hydrogel may be about 3 to about 20 kPa. Specifically, the Young's modulus value of the hydrogel may be about 5 to about 20 kPa, about 10 to about 20 kPa, or about 13 to about 17 kPa. The Young's modulus or Young modulus refers to mechanical properties that measure the stiffness of a solid material.

The hydrogel may be for ocular injection.

The hydrogel may have intraocular injectability that allows intraocular injection by using DBCO-HA.

The intraocular injectability may vary depending on the physical properties of the injected drug (viscosity, elasticity, strength, etc.), ease of preparation, transparency, safety, etc.

According to another aspect of the disclosure, provided is a drug delivery composition including the hydrogel.

According to another aspect of the disclosure, provided is an anti-inflammatory skin topical composition including the hydrogel.

According to another aspect of the disclosure, provided is a pharmaceutical composition for treating an ocular disease including the hydrogel.

The ocular disease may be a degenerative retinal disease.

The degenerative retinal disease may be one or more selected from the group consisting of Retinitis Pigmentosa (RP), Age-related Macular Degeneration (AMD), Diabetic Retinopathy, Cone Dystrophy, and Inherited Retinal Degenerations (IRD), but is not limited thereto.

The composition may selectively release the drug encapsulated within the hydrogel when exposed to an inflammatory lesion or an inflammatory environment or when in contact with an inflammatory environment.

The term “skin external application” is a concept that encompasses all compositions applied to the skin, and includes, for example, pharmaceutical compositions for topical administration, etc., including cosmetics, ointments, creams, lotions, etc., and other drugs and quasi-drugs. For example, the skin external application composition may include, but is not limited to, a cosmetic composition, a wound dressing composition, a patch dressing composition, a patch dressing sheet composition, etc.

The pharmaceutical composition may include the hydrogel and other components such as a diluent, a carrier, etc. Accordingly, the pharmaceutical composition may include, as necessary, a pharmaceutically acceptable carrier, diluent, or excipient, or a combination thereof.

Pharmaceutically acceptable carriers included in the pharmaceutical composition are those commonly used in formulation, and include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil, etc. The pharmaceutical composition may further include, in addition to the components, a lubricant, a humectant, a sweetener, a flavoring agent, an emulsifier, a suspending agent, a preservative, etc.

The pharmaceutical composition may be administered parenterally (for example, topical administration, transdermal administration, injection administration, etc.), and in the case of parenteral administration, it may be preferably administered directly to a wound, wound, or inflammation site.

The pharmaceutical composition may have intraocular injectability, which allows intraocular injection.

The wound refers to a state in which a living body is damaged, and may encompass a pathological state in which tissues forming the internal or external surface of a living body, for example, skin, muscle, nerve tissue, bone, soft tissue, internal organ, or vascular tissue, are divided or destroyed. Specifically, the wound may comprehensively refer to damage to a part of a subject such as a contusion or bruise, a laceration, an avulsion, a penetrating wound, a non-healing traumatic wound, destruction of tissue by radiation, an abrasion, a gangrene of the bone, a gunshot wound, a cut, a burn, frostbite, a skin ulcer, dry skin, keratosis, a crack, a burst, dermatitis, pain due to dermatophytosis, a surgical wound, a wound caused by vascular disease, a corneal wound, a bedsore, a wound caused by diabetes or poor circulation, a chronic ulcer, a suture site after plastic surgery, a wound caused by spinal trauma, a gynecological wound, a chemical wound, or acne, etc.

The skin regeneration may include epidermal regeneration, dermal regeneration, and regeneration of skin appendages, etc.

The appropriate dosage of the pharmaceutical composition may be prescribed in various ways depending on factors such as the formulation method, administration method, patient's age, body weight, sex, pathological condition, food, administration time, administration route, excretion rate, and response sensitivity. Meanwhile, the dosage of the pharmaceutical composition may preferably be about 0.001 mg/kg (body weight) to about 1000 g/kg (body weight) per day.

The pharmaceutical composition may be prepared in a unit dose form or may be prepared by placing it in a multi-dose container by formulating it using a pharmaceutically acceptable carrier and/or excipient according to a method that may be easily performed by a person of ordinary skill in the art to which the disclosure pertains. At this time, the formulation may be in the form of a solution, suspension or emulsion in an oil or aqueous medium, or may be in the form of an extract, powder, granule, tablet, capsule, gel, or patch, but is not limited thereto.

The term “treatment”, when used on a subject exhibiting symptoms of disease, refers to stopping, delaying, or alleviating the progression of the disease.

The same parts as described in the hydrogel equally apply to the drug delivery composition, the skin external application composition and the pharmaceutical composition.

According to another aspect of the disclosure, provided is a method of treating an ocular disease, including administering to a subject a therapeutically effective amount of the hydrogel, wherein the hydrogel is a hydrogel formed by cross-linking hyaluronic acid with a crosslinker, a drug is encapsulated within the hydrogel, and the crosslinker includes a peptide that is degradable by cathepsins.

The hyaluronic acid may be DBCO-conjugated hyaluronic acid (DBCO-HA).

The DBCO-HA may be a product in which the carboxyl group of hyaluronic acid and DBCO are linked by copper-free click chemistry.

The DBCO-HA may be a product in which the carboxyl group of hyaluronic acid and DBCO are linked by copper-free click chemistry.

The hydrogel may selectively release the drug at the inflammatory lesion.

According to an aspect of the disclosure, the drug may be, but is not limited to, an enhancer of zeste homolog 2 (EZH2) inhibitor.

The hydrogel may be for ocular injection.

The ocular disease may be a degenerative retinal disease.

The degenerative retinal disease may be one or more selected from the group consisting of Retinitis Pigmentosa (RP), Age-related Macular Degeneration (AMD), Diabetic Retinopathy, Cone Dystrophy, and Inherited Retinal Degenerations (IRD), but is not limited thereto. The same aspects as described in the hydrogel apply equally to the ocular disease treatment method.

According to another aspect of the disclosure, provided is a method of preparing a hydrogel, including (1) a first step of preparing dibenzylcyclooctyne (DBCO)-conjugated hyaluronic acid (DBCO-HA), (2) a second step of mixing the DBCO-HA with a solvent, (3) a third step of mixing a drug, and (4) a fourth step of adding a crosslinker, wherein the crosslinker includes a peptide degradable by cathepsins.

The first step may include dissolving hyaluronic acid in a mixed solution of DMSO (Dimethyl Sulfoxide) and distilled water.

The first step may include dissolving 1-ethyl-3-(3-dimethylaminopropyl) carbodiimide hydrochloride (EDC) in a hyaluronic acid solution.

The first step may include dissolving N-hydroxysuccinimide (NHS) in a hyaluronic acid solution.

The first step may include adding DBCO-PEG4-amine to a hyaluronic acid solution.

According to an aspect of the disclosure, the first step may include a first dissolution step of dissolving hyaluronic acid in a mixed solution of dimethyl sulfoxide (DMSO) and distilled water, a second dissolution step of dissolving 1-ethyl-3-(3-dimethylaminopropyl) carbodiimide hydrochloride (EDC), a third dissolution step of dissolving N-hydroxysuccinimide (NHS), and an addition step of adding DBCO-PEG4-amine.

According to an aspect of the disclosure, the peptide that is degraded by the cathepsin may include an amino acid sequence of SEQ ID NO: 1.

According to an aspect of the disclosure, the drug may be an enhancer of zeste homolog 2 (EZH2) inhibitor.

According to an aspect of the disclosure, the solvent of the second step may be, but is not limited to, deionized water (DI water).

In the third step, the average molecular weight of the drug may be about 600 to about 10,000 g·mol⁻¹. Specifically, the average molecular weight of the drug may be about 600 to about 8,000 g·mol⁻¹ or about 600 to about 5,000 g·mol⁻¹.

According to an aspect of the disclosure, when the average molecular weight of the drug is less than about 600 g·mol⁻¹, the drug-encapsulated hydrogel prepared by the method may release the drug not only in an inflammatory environment where cathepsin exists but also in a normal environment, so that the specificity for an inflammatory condition in drug release may decrease, and when the average molecular weight of the drug is more than about 10,000 g·mol⁻¹, the drug-encapsulated hydrogel prepared by the method may have a decreased drug encapsulation efficiency or a decreased release amount of the encapsulated drug, so that the anti-inflammatory effect may decrease.

In the fourth step, the crosslinker may be mixed into the hydrogel at a molar concentration of about 3 mM or more, about 3 to about 50 mM, about 3 to about 40 mM, about 3 to about 30 mM, about 3 to about 20 mM, about 3 to about 10 mM, or about 3 to about 5 mM.

According to an aspect of the disclosure, when the molar concentration of the crosslinker included in the hydrogel prepared by the method is less than about 3 mM, the mechanical properties of the hydrogel, such as network density and hardness, etc., decrease, so that the hydrogel may release drugs not only in an inflammatory environment in which cathepsin exists but also in a normal environment, and thus the specificity for an inflammatory condition in drug release may decrease, and when the molar concentration of the crosslinker included in the hydrogel prepared by the method exceeds about 50 mM, the hydrogel may have a decreased drug encapsulation efficiency or a decreased release amount of the encapsulated drug, so that the anti-inflammatory effect may decrease.

The same aspects as described in the above hydrogel apply equally to the method.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A illustrates measurement of an mRNA expression level of an inflammatory marker at postnatal week 3 (PW3) and postnatal week 7 (PW7) in normal (wild-type, wt) and retinal degeneration (retinal degeneration 10, rd10) mouse models; (Mean±SD; n=3)

FIG. 1B illustrates measurement of an mRNA expression level of cathepsins L, S, and B at postnatal week 3 (PW3) and postnatal week 7 (PW7) in normal (wt) and retinal degeneration (rd10) mouse models; (Mean±SD; n=3)

FIG. 1C illustrates measurement of an activity of cathepsins L, S, and B at postnatal week 3 (PW3) and postnatal week 7 (PW7) in normal (wt) and retinal degeneration (rd10) mouse models; (Mean±SD; n=3)

FIG. 1D is a diagram illustrating a result of immunofluorescence staining of inflammatory markers at postnatal week 3 (PW3) and postnatal week 7 (PW7) in normal (wt) and retinal degeneration (rd10) mouse models;

FIG. 1E is a diagram illustrating a result of immunofluorescence staining of cathepsins L, S, and B at postnatal week 3 (PW3) and postnatal week 7 (PW7) in normal (wt) and retinal degeneration (rd10) mouse models;

FIG. 2A is a schematic diagram illustrating a preparation process of an inflammatory responsive hydrogel (IRH);

FIG. 2B is a diagram illustrating that a rate of IRH degradation varies depending on an amount of cathepsin secreted in an inflammatory microenvironment;

FIG. 3A is a schematic diagram of a structure of a hydrogel (IRH) cross-linked by a connection of a cathepsin-cleavable peptide crosslinker and DBCO-HA of an aspect;

FIG. 3B is a diagram illustrating a result of analyzing a chemical component of the prepared DBCO-HA by using 1H-NMR;

FIG. 3C illustrates measurement of a storage modulus (G′; solid symbols) and loss modulus (G″; open symbols) of IRH at four different concentrations (0.25, 0.5, 1, and 2 mM) of a cathepsin-degradable crosslinker;

FIG. 3D illustrates a measurement of a Young's modulus value of IRH at three different concentrations (0.25, 0.5, and 1 mM) of a cathepsin-degradable crosslinker;

FIG. 3E is a diagram illustrating a scanning electron microscopy (SEM) measurement of IRH at three different concentrations (0.25, 0.5, and 1 mM) of a cathepsin-degradable crosslinker;

FIG. 3F illustrates measurement of a permeability value of IRH at three different concentrations (0.25, 0.5, and 1 mM) of a cathepsin-degradable crosslinker;

FIG. 3G and FIG. 3H illustrate measurement of cytotoxicity of IRH at three different concentrations (0.25, 0.5, and 1 mM) of a cathepsin-degradable crosslinker;

FIG. 4A is a diagram illustrating a result of measuring fluorescence emitted by IRH loaded with dextran of an aspect, a one-dimensional fluorescent dye, after incubation in four different solutions: phosphate buffered saline (PBS), control medium (Media), inactivated microglia conditioned medium (NM CM), and activated microglia conditioned medium (AM CM), using IVIS, and FIG. 4B is a diagram quantifying results of the fluorescence measurement;

FIG. 4C is a diagram illustrating results of measuring a release level of an EZH2 inhibitor after exposing IRH loaded with an EZH2 inhibitor of an aspect to control medium (Media), inactivated microglia conditioned medium (NM CM), and activated microglia conditioned medium (AM CM), respectively;

FIG. 4D is a diagram illustrating results of measuring a release level of an EZH2 inhibitor after culturing IRH loaded with an EZH2 inhibitor of an aspect in a medium without cathepsin, a medium including 10 ng/ml cathepsin, and a medium including 100 ng/ml cathepsin;

FIG. 5A is a diagram illustrating results of measuring mRNA levels of TNF-α, IL1β, CCL2, and CCL5 in microglia activated by induced inflammation;

FIG. 5B is a diagram illustrating results of measuring protein activity levels of cathepsins L, S, and B in microglia activated by induced inflammation;

FIG. 5C is a diagram illustrating results of measuring mRNA levels of inflammatory factors after culturing inflammatory microglia in four different conditions of {1) medium only (Control), 2) hydrogel only (Gel only), 3) EZH2 inhibitor only (Drug only), and 4) hydrogel loaded with EZH2 inhibitor (Drug & Gel)};

FIG. 5D is a diagram illustrating results of measuring expression levels of p-STAT1, STAT1, and IRF1 after culturing inflammatory microglia in four different conditions of {1) medium only (Control), 2) hydrogel only (Gel only), 3) EZH2 inhibitor only (Drug only), and 4) hydrogel loaded with EZH2 inhibitor (Drug & Gel)};

FIG. 5E is a schematic diagram illustrating that an IRH loaded with EZH2 inhibitor exhibits anti-inflammatory effects by reducing IRF1/STAT1 signaling that regulates inflammation;

FIG. 6A is a diagram illustrating results of measuring mRNA levels of inflammatory factors in a retina after injection of three different groups of {1) hydrogel without drug (Gel only group; n=6), 2) EZH2 inhibitor in a form of a solution without hydrogel (Drug only group; n=6), and 3) hydrogel loaded with EZH2 inhibitor (Drug & Gel group; n=6)} into the vitreous body of rd10 mice, an RP model;

FIG. 6B is a diagram illustrating results of immunofluorescence staining of microglia (Iba1) and an inflammatory marker (IL1B) in the retina after injection of three different groups of {1) hydrogel without drug (Gel only group; n=6), 2) EZH2 inhibitor in the form of a solution without hydrogel (Drug only group; n=6), and 3) hydrogel loaded with EZH2 inhibitor (Drug & Gel group; n=6)} into the vitreous body of rd10 mice, an RP model {ONL outer nuclear layer (photoreceptor cell bodies), OPL outer plexiform layer, INL inner nuclear layer (bipolar cell bodies), IPL inner plexiform layer, GCL ganglion cell layer}, and FIG. 6C is a diagram quantifying the results;

FIG. 6D is a diagram illustrating results of immunofluorescence staining of retinal microglia (Iba1) and a phagocyte marker (CD68) after injection of three different groups of {1) hydrogel without drug (Gel only group; n=6), 2) EZH2 inhibitor in the form of a solution without hydrogel (Drug only group; n=6), and 3) hydrogel loaded with EZH2 inhibitor (Drug & Gel group; n=6)} into the vitreous body of rd10 mice, an RP model, and FIG. 6E is a diagram quantifying the results;

FIG. 6F is a diagram illustrating results of immunofluorescence staining of cone cells in the retina after injection of three different groups of {1) hydrogel without drug (Gel only group; n=6), 2) EZH2 inhibitor in the form of a solution without hydrogel (Drug only group; n=6), and 3) hydrogel loaded with EZH2 inhibitor (Drug & Gel group; n=6)} into the vitreous body of rd10 mice, an RP model, and FIG. 6G is a diagram quantifying the results;

FIG. 6H illustrates measurements of the thickness of an outer nuclear layer (ONL) after injection of three different groups of {1) hydrogel without drug (Gel only group; n=6), 2) EZH2 inhibitor in the form of a solution without hydrogel (Drug only group; n=6), and 3) hydrogel loaded with EZH2 inhibitor (Drug & Gel group; n=6)} into the vitreous body of rd10 mice, an RP model; (Mean±SD; n=3)

FIG. 7A is a diagram illustrating results of recording light-induced responses in retinal ganglion cells (RGCs) of untreated rd10 mice and rd10 mice treated with Gel only, Drug only, and Drug & Gel to investigate the potential delay in physiological visual function loss after treatment with IRH;

FIG. 7B and FIG. 7C are diagrams illustrating results of measuring the number of spikes (FIG. 7B) and peak firing rate (FIG. 7C) of light responses in RGCs of untreated rd10 mice and rd10 mice treated with Gel only, Drug only, and Drug & Gel;

FIG. 7D is a diagram illustrating results of measuring a spontaneous peak firing rate in response to light in RGCs of untreated rd10 mice and rd10 mice treated with Gel only, Drug only, and Drug & Gel;

FIG. 8A is a diagram illustrating spike activity induced by seven repetitions of the same light stimulus in RGCs of untreated rd10 mice and rd10 mice treated with Gel only, Drug only, and Drug & Gel; and

FIG. 8B is a diagram illustrating a matrix representation of a spike time tiling coefficients (STTC) of spikes induced by seven repetitions of the same light stimulus in RGCs of untreated rd10 mice and rd10 mice treated with Gel only, Drug only, and Drug & Gel, FIG. 8C is a diagram illustrating percentages of defined and undefined STTC values in each group, and FIG. 8D is a diagram illustrating a STTC distribution of each group as a violin plot.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the disclosure will be described in more detail by the following examples. However, these examples are only intended to illustrate the disclosure, and the scope of the disclosure is not limited by these examples.

### Example 1. Preparation of a Hydrogel (IRH) Enabling Drug Release in Response to Disease Conditions

### 1-1. Confirmation of Cathepsin Overexpression Induced by Inflammatory Activity in Retinal Degeneration

To identify target biomarkers for Inflammatory Responsive Hydrogel (IRH), biomolecules that are increased by inflammation as retinal degeneration (RD) progresses were first explored.

For example, cathepsin enzymes have been proven to be overexpressed in an inflammatory environment such as skin wounds, rheumatoid arthritis, and inflammatory brain diseases. Among immune cells, macrophages exhibit a phenotype of inflammatory M1 macrophages in inflammatory environments and secrete high levels of cathepsins. Cathepsins are cysteine proteases that play a key role in physiological processes of various inflammatory diseases. In addition, secreted cathepsins degrade an extracellular matrix, and a proteolytic ability of cathepsins allows the development of cathepsin-sensitive hydrogels by generating protein crosslinkers that are degraded by cathepsins. Because various cathepsin isoforms are simultaneously oversecreted in an inflammatory microenvironment, a cathepsin-responsive hydrogel that reacts to various cathepsin enzymes was designed.

To test whether cathepsin may serve as a biomarker of IRH in RD, levels of several inflammatory markers and cathepsin isoforms were analyzed in the retinas of rd10 mice, a well-established Retinitis Pigmentosa (RP) model. First, the mRNA expression level of six representative inflammatory markers were measured, namely tumor necrosis factor (TNF)-α, interleukin 1β (IL1β), C-C motif chemokine ligand 2 (CCL2), C-C motif chemokine ligand 5 (CCL5), T-lymphocyte activating antigen CD86, and cluster of differentiation 68 (CD68). Animals were sacrificed at postnatal week (PW) 3 or week 7, corresponding to the early and middle stages of RD, respectively; age-matched normal (wt) mice were also used as a control group.

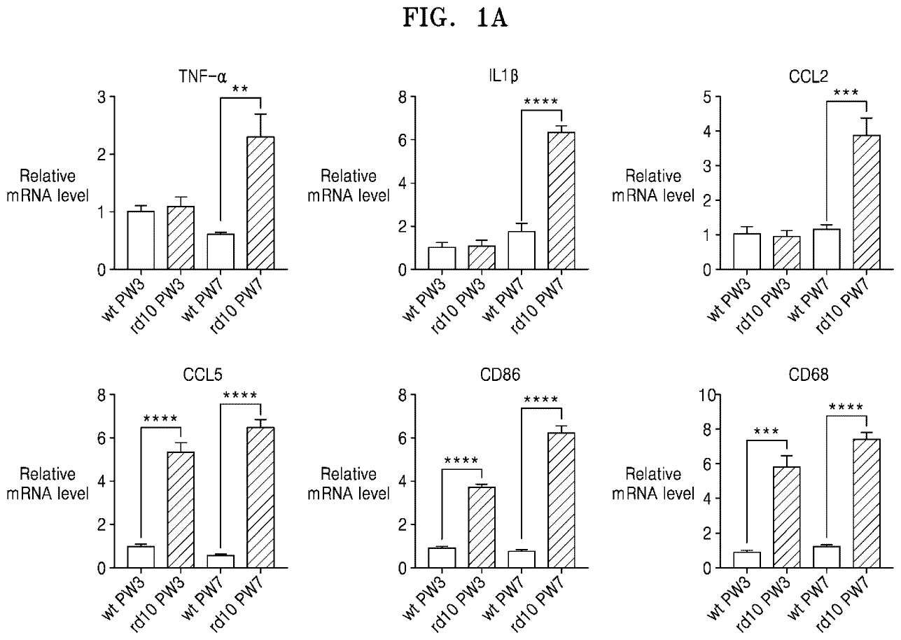

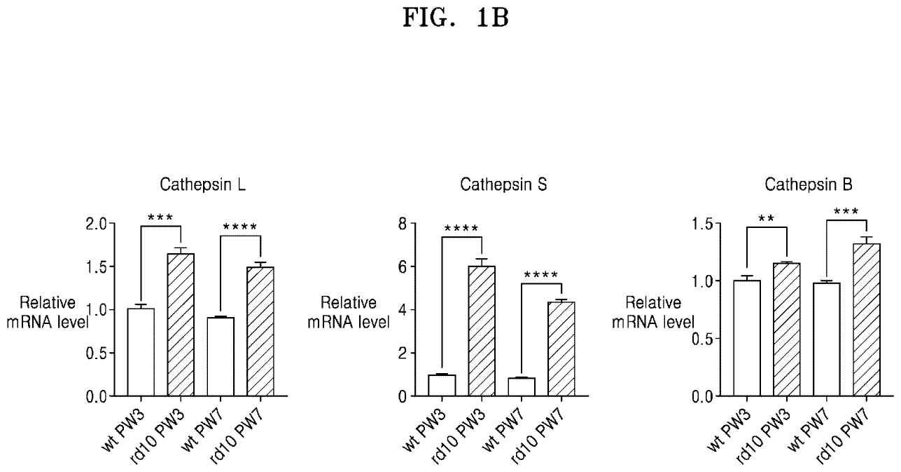

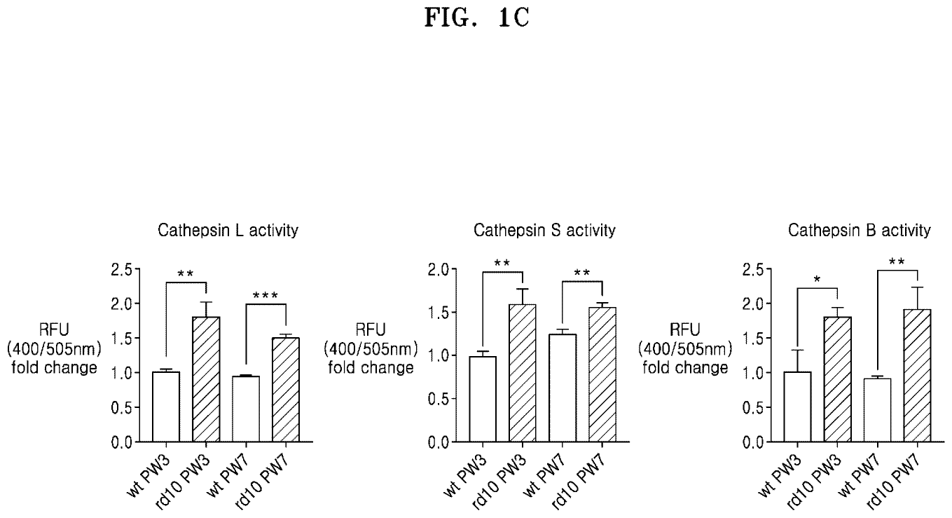

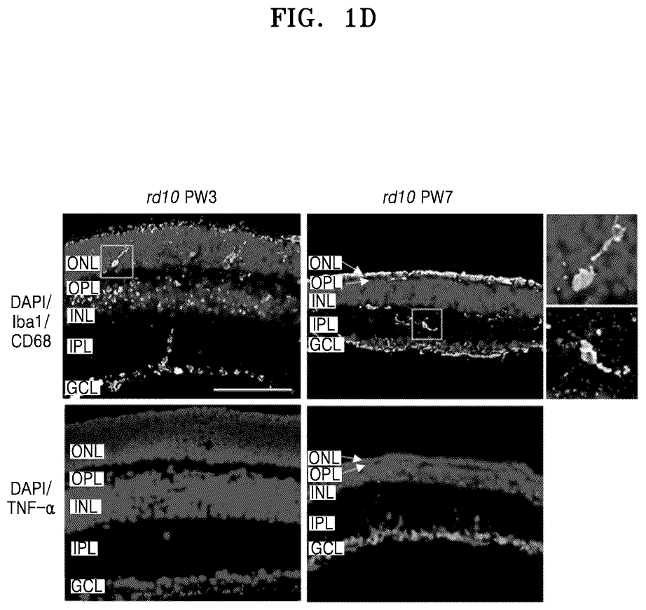

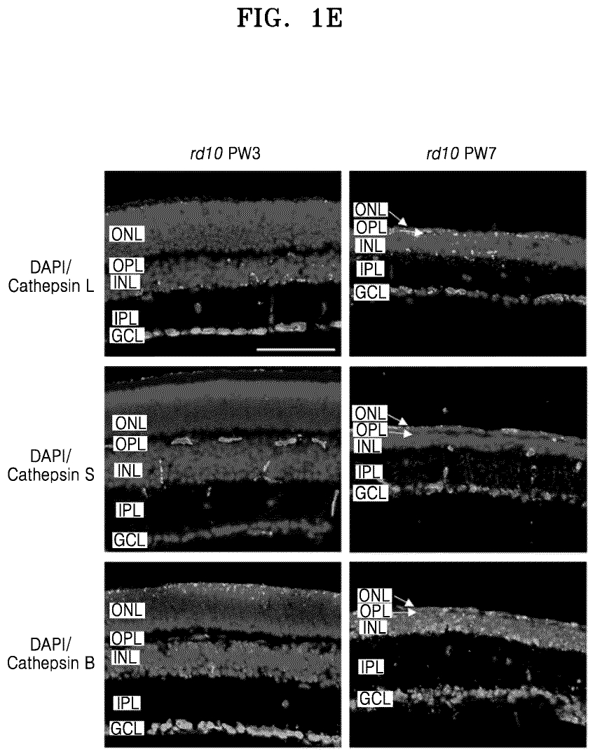

At PW3, the first three markers (in other words, TNF-α, IL1β, and CCL2) were not increased, whereas the other three markers (in other words, CCL5, CD86, and CD68) showed significantly increased mRNA levels even at this early RD stage (FIG. 1A). Expression levels of all inflammatory factors increased as the disease progressed to age PW7 in rd10 mice (FIG. 1A). These results clearly demonstrate that RP involves inflammatory activity.

In relation to the inflammatory response, an mRNA and protein activity level of three subtypes of cathepsin enzymes (in other words, L, S, and B) were also up-regulated in rd10 mice at PW3 (FIG. 1B and FIG. 1C).

In addition to mRNA level analysis and protein activity assays, immunofluorescence staining of the aforementioned biomolecules was additionally performed. Notably, CD68, a phagocytic function of inflammatory microglia (Iba1+CD68+) in a retina known to accelerate photosensitive cell degeneration, was detected in an outer nuclear layer (ONL) and inner plexiform layer (IPL) at PW3 and PW7, respectively, of rd10 animals (upper row of FIG. 1D). In addition, TNF-α, another inflammatory marker, was found in a ganglion cell layer (GCL) (lower line of FIG. 1D), consistent with an mRNA level result showing increased levels in PW7 animals (compared with the leftmost lower panel of FIG. 1A). Interestingly, in a result of Immunofluorescence staining, cathepsins were observed in more retinal layers (FIG. 1E). For example, all cathepsin isoforms were weakly expressed in an outer part of the photoreceptor (in other words, just above the ONL) in PW3 of animals (left column of FIG. 1E). Strong bands of cathepsins L and B were also found at this early stage of RD. Then, expression of all three isoforms appears to become widespread as RD progresses (right column of FIG. 1E). Overall, the inflammatory environment induced by inherited RD resulted in cathepsin overexpression.

These results suggest that targeting cathepsins may lead to the development of inflammatory responsive hydrogels, which may also be applied to the treatment of RP.

### 1-2. Preparation of DBCO-Conjugated Hyaluronic Acid (DBCO-HA)

Hyaluronic acid (HA, 2 M Da; SK Bioland, Seoul, Korea) was dissolved in a DMSO:distilled water (1:1) solution at a concentration of 0.5 wt %. Then, 0.6 mM 1-ethyl-3-(3-dimethylaminopropyl) carbodiimide hydrochloride (EDC) was dissolved in the HA solution, followed by 0.6 mM N-hydroxysuccinimide (NHS) dissolved in the solution for 30 minutes. 0.4 mM DBCO-PEG4-amine (#A103P, Click Chemistry Tools, Scottsdale, USA) was slowly added to the prepared solution. The reaction was maintained at room temperature for 24 hours. After the reaction was completed, the solution was transferred to a 25 kDa molecular weight cutoff (MWCO) dialysis tube and dialyzed in deionized water (DI) for 3 days. The dialysis solution was replaced every 10 hours. DBCO-conjugated HA (DBCO-HA) solution was sterilized with a 0.22 μm syringe filter and lyophilized for 3 days. Successful conjugation of DBCO and HA was confirmed by proton nuclear magnetic resonance (1H-NMR) spectroscopy (a sample concentration of DBCO-HA solution was prepared at 2 wt % in deuterium oxide).

### 1-3. Preparation of DBCO-HA Hydrogel (IRH) Enabling Disease-Responsive Release of Anti-Inflammatory Agents

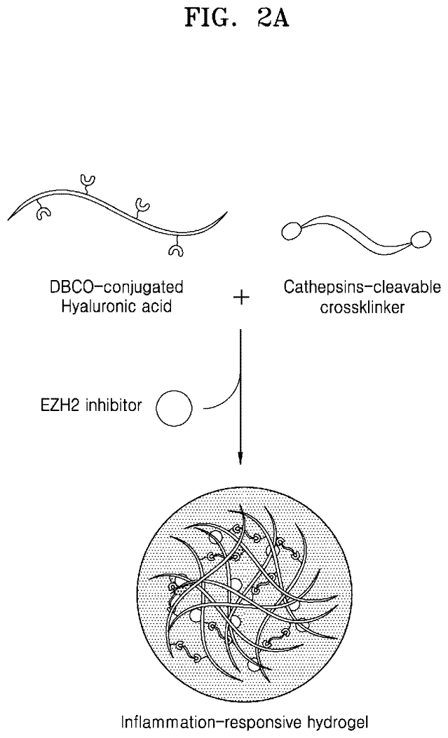

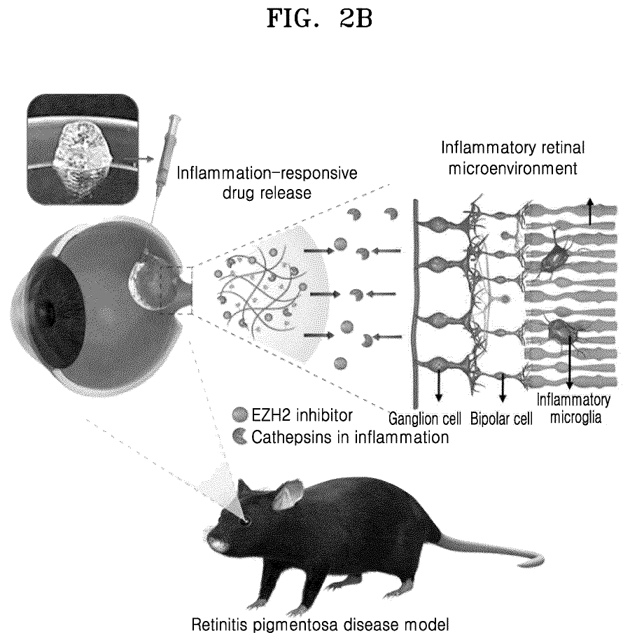

On-demand drug delivery has been proven, that it may not only reduce an adverse effect of overdose, but also enhance therapeutic effect by optimizing an amount of drug delivered, depending on the severity of a target disease. IRH was prepared through cross-linking between DBCO-conjugated HA and a cathepsin-cleavable crosslinker. In addition, an EZH2 inhibitor was encapsulated when a hydrogel was cross-linked (FIG. 2A). The degradation rate of the IRH was designed to vary depending on an amount of cathepsins secreted in an inflammatory microenvironment. The prepared hydrogel including the drug may be intravitreally injectable into the eye (FIG. 2B). IRH was degraded by cathepsins oversecreted in an inflammatory retinal microenvironment, releasing a preloaded EZH2 inhibitor. Accordingly, the hydrogel effectively alleviated inflammation by delivering the EZH2 inhibitor as needed in response to inflammatory activity occurring in the early stage of RP.

The cathepsin-cleavable peptide crosslinker used was azidoacetyl-ARLRK-azidoacetyl (MW: 808.51 Da, purity: 99%; model number, Peptron, Daejeon, Korea). After dissolving 2 wt % DBCO-HA in DI water, cathepsin-cleavable crosslinker was added at various concentrations: 0.25 mM, 0.5 mM, and 1 mM solution mixtures were incubated at 37° C. for 30 minutes.

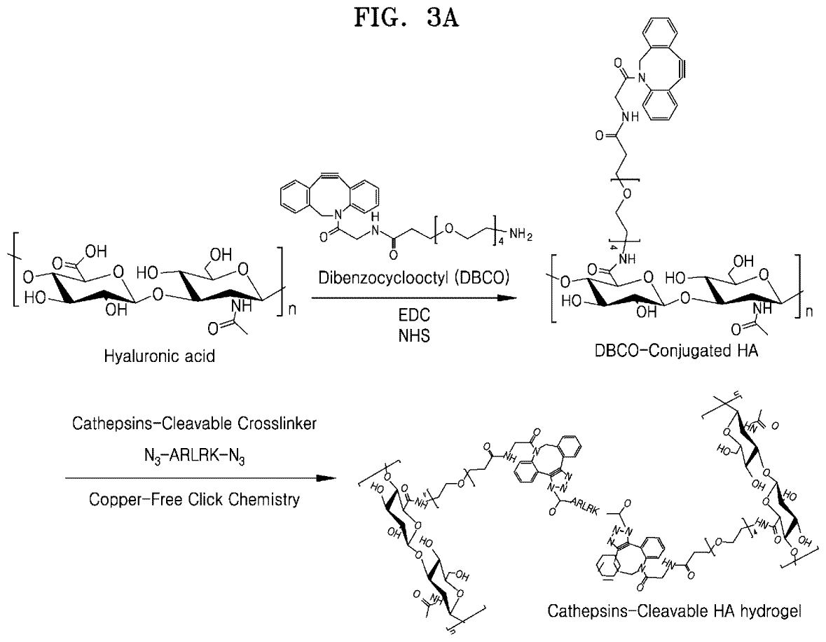

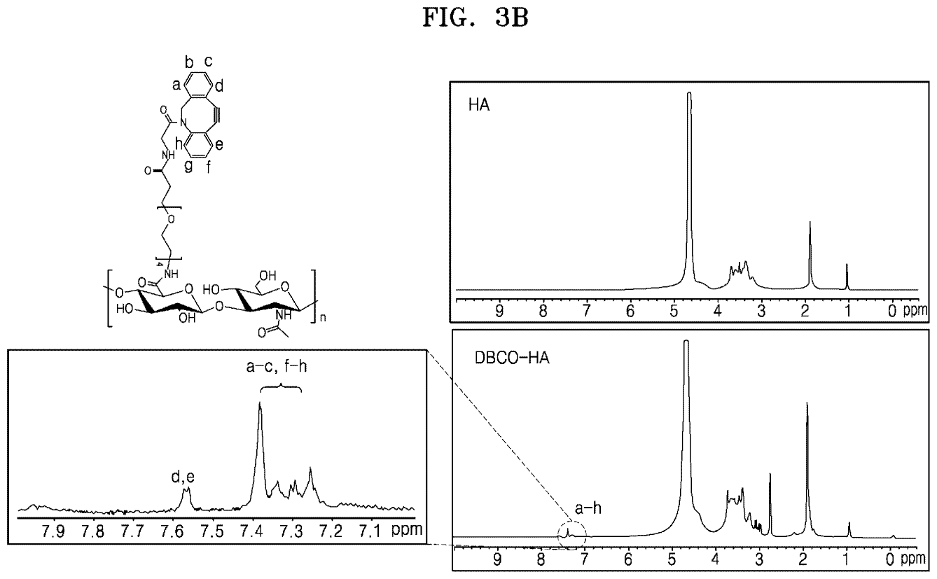

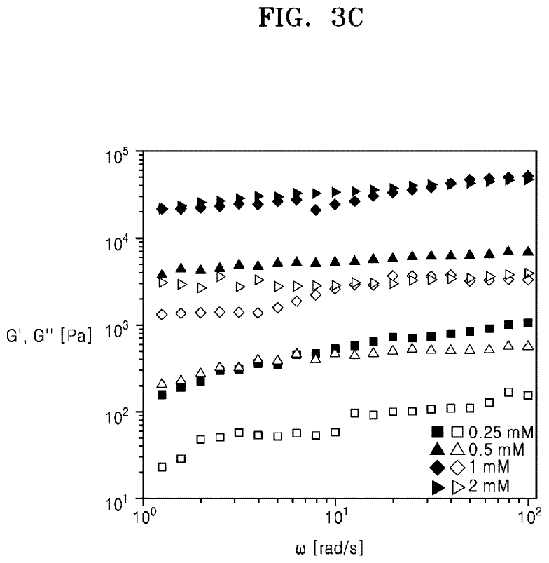

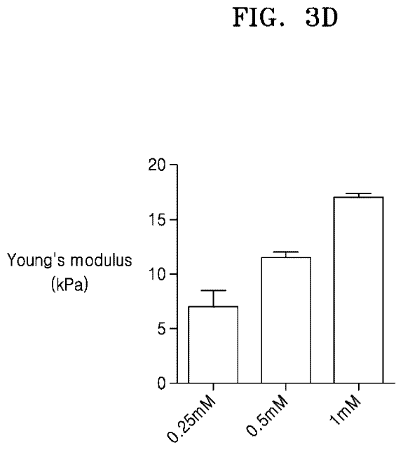

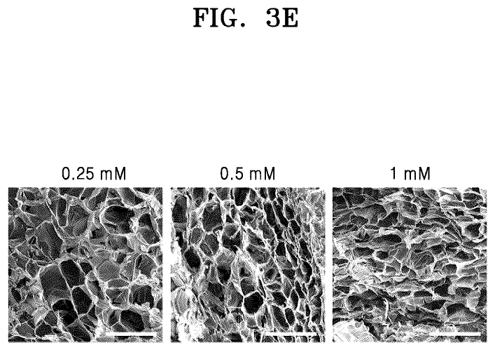

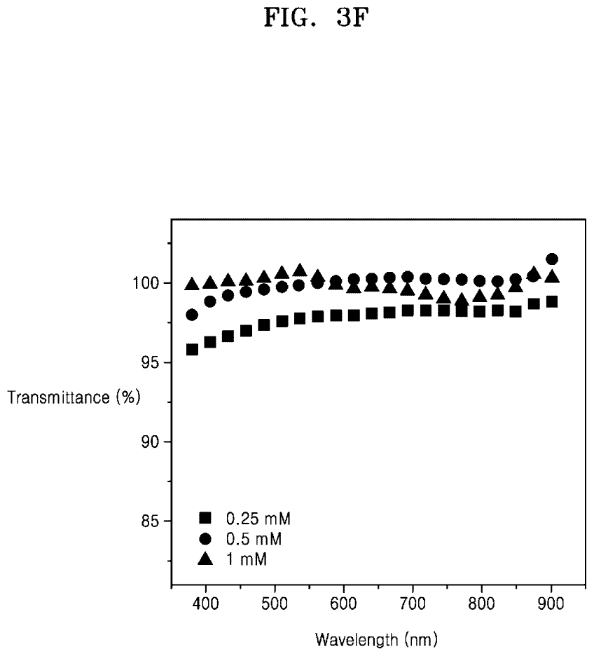

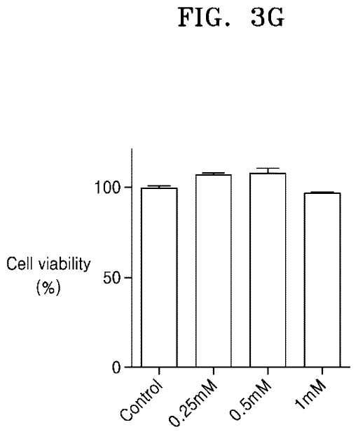

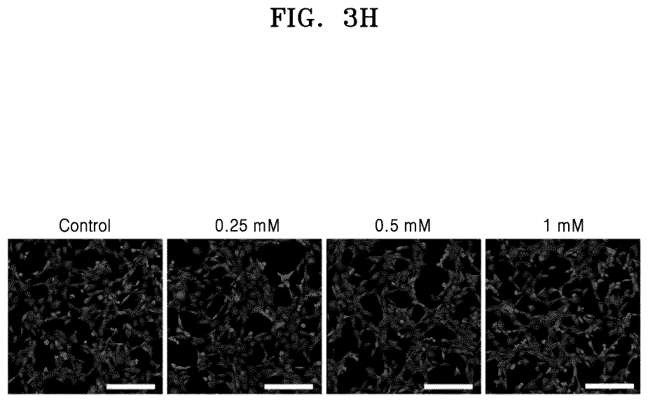

Injectable IRH was prepared by a copper-free click chemistry between the DBCO of HA and an azide of a cathepsin-cleavable crosslinker (c-c crosslinker) (FIG. 3A). Successful conjugation of DBCO to HA was confirmed using 1H-NMR: several peaks representing DBCO were detected between about 7 ppm and about 8 ppm (see enlarged inset on the left side of FIG. 3B).

### Experimental Example 1. Characteristic Analysis of IRH

### 1-1. Analysis of Mechanical Properties

The mechanical properties of IRH were analyzed using a rheometer. As a result, both a storage modulus (G′) and a loss modulus (G″) increased as a c-c crosslinker increased, but were saturated at a concentration of 2 mM (FIG. 3C). Since the storage modulus (G′) represents the elasticity and the loss modulus (G″) represents the viscosity of the material, an increase in both parameters implies an acceleration in a viscoelasticity of the IRH. The stiffness of IRH was measured by Young's modulus and was found to increase as a concentration of c-c crosslinker increased, with an average modulus of ˜18 kPa when 1 mM c-c crosslinker was used (FIG. 3D). Given the low modulus of the vitreous humor (typically known to be a few Pa, but varies across studies), adverse effects due to mechanical mismatch between a hydrogel and a vitreous body are possible and should be investigated in future studies. As shown in a scanning electron microscope (SEM) image, an internal network structure of the hydrogel became denser with increasing c-c crosslinker concentration (FIG. 3E). A denser internal network refers to slower biodegradation, increasing a residence time of the hydrogel at a disease site.

### 1-2. Transparency Measurement

Since the most important characteristic of intraocular injection materials is transparency, a transmittance of the prepared IRH was measured. Transparency was about 98% to about 100% at all c-c crosslinker concentrations tested (in other words, 0.25, 0.5, and 1 mM), indicating that IRH will not interfere with a residual vision of RP targets when used clinically in the future (FIG. 3F).

### 1-3. Cytotoxicity Analysis

Finally, cell viability was validated by examining in vitro cytotoxicity of IRH using a Live & Dead/Cytotoxicity Assay Kit (#L3224, Invitrogen, Waltham, Massachusetts, USA). The results clearly showed that the hydrogels were non-toxic for various c-c crosslinker concentrations (FIG. 3G and FIG. 3H). All these results indicate that the IRH has mechanical, optical, and biocompatible properties suitable for intraocular implantation.

### Experimental Example 2. Confirmation of Inflammation-Responsive Drug Release by IRH

### 2-1. Demonstration of Inflammation-Specific Drug Release Via Enzyme Activation

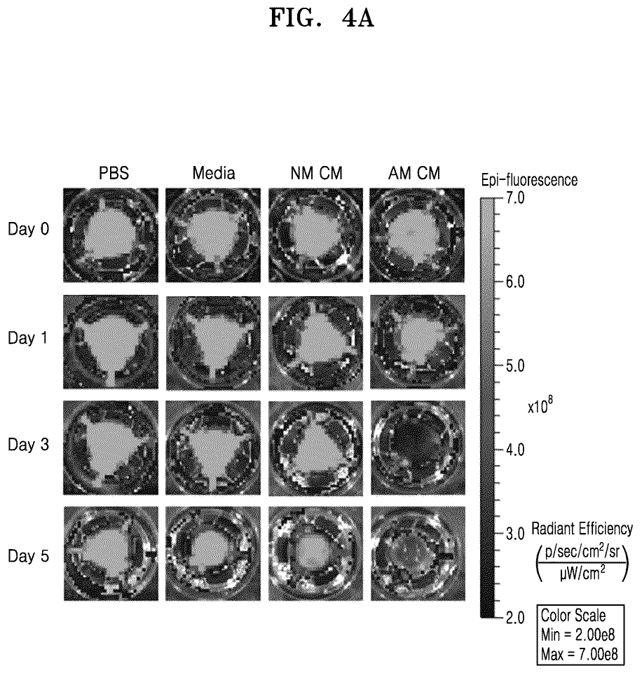

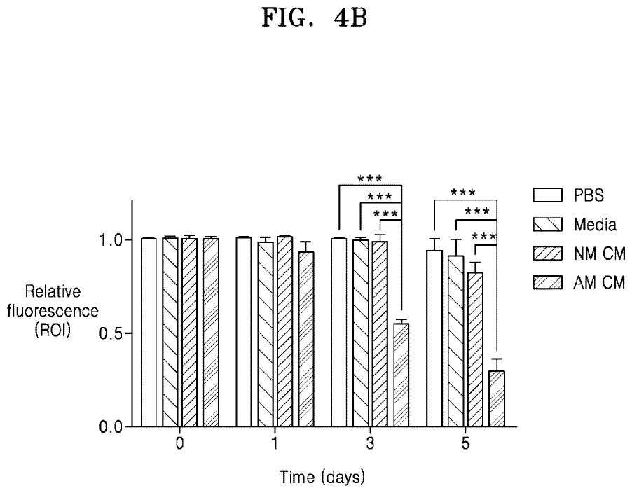

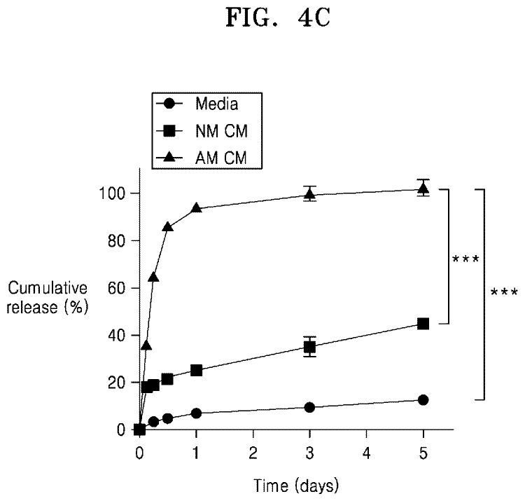

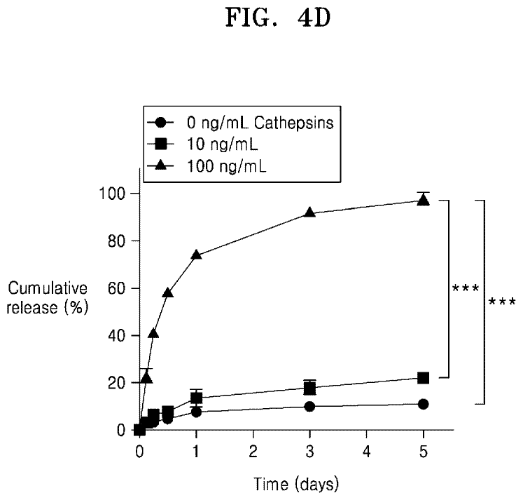

To test an inflammatory reactivity of a hydrogel, dextran fluorescent dye and small molecule encapsulated hydrogels were cultured together with inflammatory microglia conditioned medium and cathepsins. The release of dextran from hydrogels prepared under the same conditions (c-c crosslinker concentration 1 mM and other condition parameters) was maintained for 5 days in four different solutions, phosphate buffered saline (PBS), control medium (Media), inactivated microglia conditioned medium (NM CM), and activated microglia conditioned medium (AM CM). Therefore, AM CM was assumed to reflect an inflammatory microenvironment of RP where inflammatory microglia is present. The amount of dextran released from each hydrogel was visualized using an in vivo imaging system (IVIS) (FIG. 4A) and summarized in a bar graph format (FIG. 4B). As shown in both plots (FIG. 4A and FIG. 4B), IRH exhibited rapid release properties in activated microglial cell medium. Dextran intensity significantly decreased in the AM CM group on day 3 (0.55±0.03) and day 5 (0.29±0.07), but the NM CM group showed no significant decrease on day 3 (0.98±0.04) and day 5 (0.82±0.06). These results indicate that a dye was rapidly released by a degraded IRH network in an inflammatory microenvironment. Hydrogels encapsulated with small molecule EZH2 inhibitors were cultured together with conditioned medium, a microglial cell culture medium, for 1 day. Consistent with the IVIS results (FIG. 4A), the release rate of the EZH2 inhibitor was accelerated in activated microglia conditioned medium (AM CM) (FIG. 4C), suggesting that a drug may be released as needed depending on the severity of inflammation. However, the EZH2 inhibitor had a smaller molecular weight and the release rate was faster than the dextran dye. Additionally, relevant inflammatory enzymes (in other words, cathepsins L, S, and B) increased the release of EZH2 inhibitors as cathepsin concentration increased (FIG. 4D). In the cathepsin-free control group, only ˜10% of the EZH2 inhibitor was released over 5 days, whereas 10 ng/ml cathepsin induced ˜20% of EZH2 inhibitor release. Furthermore, in the case of 100 ng/ml cathepsin, ˜80% of the EZH2 inhibitor was released on the first day and 100% release was reached by day 5. Overall, IRH exhibited the ability to release active drugs in response to inflammatory conditions. Although repeated injections of EZH2 inhibitors together with IRH may be required in clinical cases depending on recurrence of inflammation, the frequency of injections may be significantly reduced due to the inflammatory reactivity of the hydrogel.

2-2. Demonstration of the Anti-Inflammatory Effect of IRH Loaded with EZH2 Inhibitor on Inflammatory Microglia

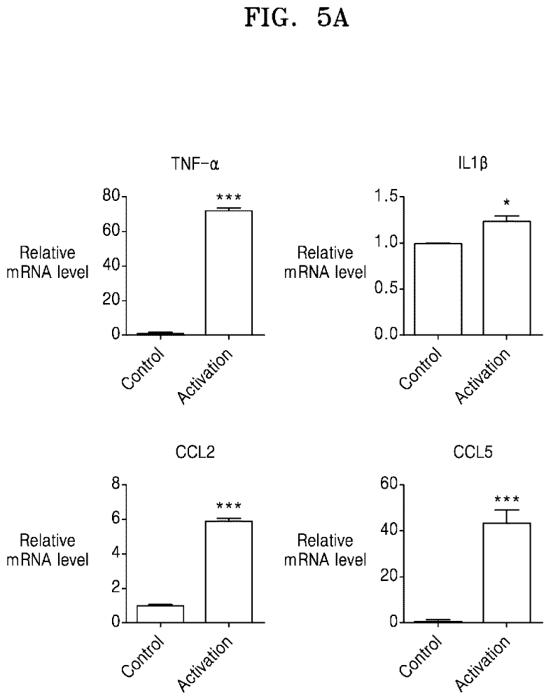

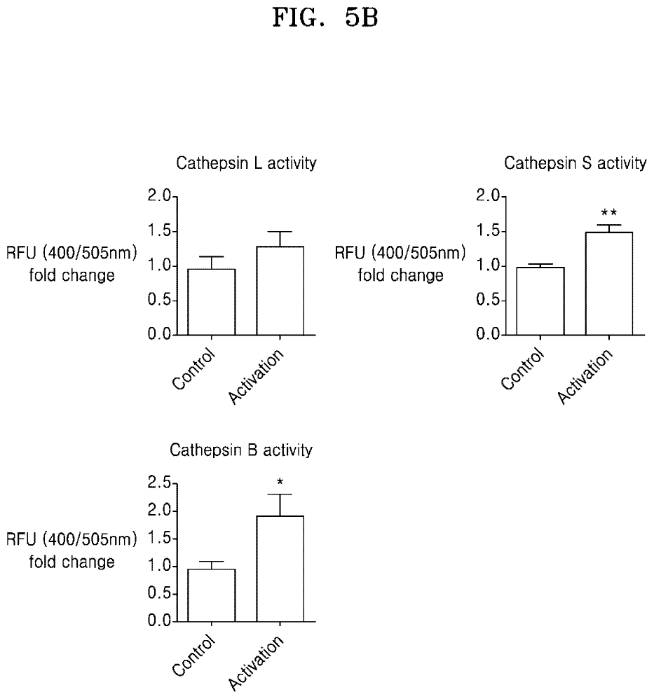

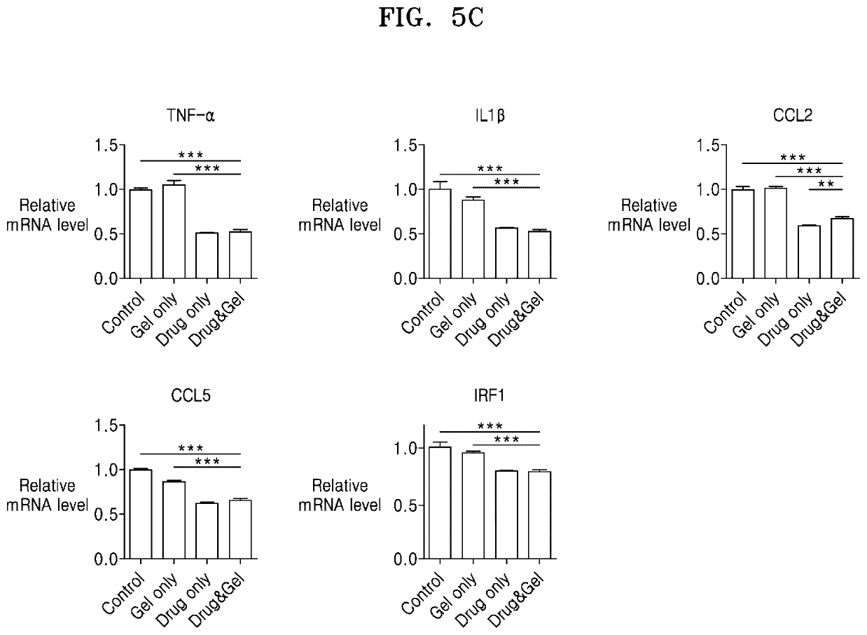

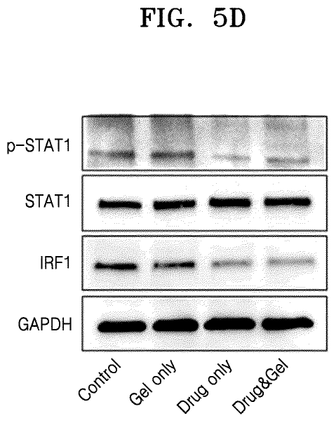

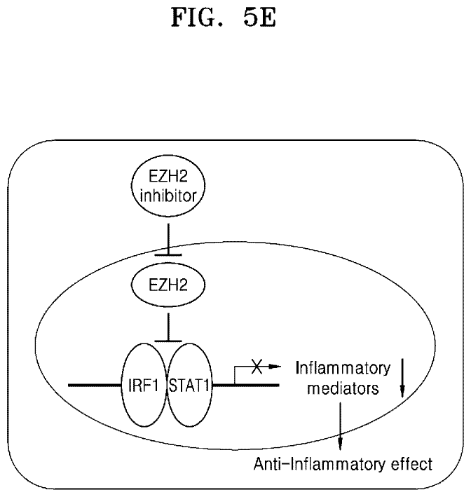

Epigenetic regulation of a retinal microenvironment has been studied as a promising therapeutic strategy in various retinal degenerative diseases. For example, since EZH2 is known to contribute to rod cell photoreceptor death and microglial inflammation, selectively regulating EZH2 in a controlled manner may be a beneficial approach for safe and effective therapeutic outcomes in retinal degenerative diseases associated with inflammatory activity. Before conducting in vivo experiments, in vitro experiments were performed to confirm the anti-inflammatory effect of IRH loaded with an EZH2 inhibitor on inflammatory microglia. To test the anti-inflammatory effect of EZH2-loaded IRH, microglia was converted into inflammatory microglia by applying 50 ng/mL TNF-α to the medium and then analyzed various inflammatory activation markers. mRNA levels of TNF-α, IL1β, CCL2, and CCL5 were up-regulated in activated microglia due to induced inflammation (FIG. 5A). Consistent with the up-regulated inflammatory marker, protein activity levels of cathepsins L, S, and B were increased (FIG. 5B). To test the anti-inflammatory effect of the EZH2 inhibitor, inflammatory microglia were first treated with an EZH1/2 inhibitor (Valemetostat) and the EZH2 inhibitor (Tazemetostat). Both EZH2 inhibitor drugs effectively suppressed the inflammatory response of microglia. Based on the results, Tazemetostat was selected for the selective inhibition of EZH2. Then, to investigate the transient anti-inflammatory effect of IRH, inflammatory microglia were cultured for 1 day under four different conditions: 1) medium only (Control), 2) hydrogel only (Gel only), 3) EZH2 inhibitor only (Drug only), and 4) hydrogel loaded with EZH2 inhibitor (Drug & Gel). As expected, both the Drug only group and the Drug & Gel group effectively reduced the inflammatory response in terms of mRNA expression levels (red and green bars in FIG. 5C). It is worth noting that the EZH2 inhibitor loaded into the hydrogel (in other words, Drug & Gel group) did not lose drug activity and showed anti-inflammatory effects similar to those treated with only the EZH2 inhibitor (in other words, Drug only group). No statistical differences were found between the two groups except for CCL2 (compare red and green bars in FIG. 5C). Accordingly, delivery of EZH2 inhibitors via IRH may be an effective anti-inflammatory therapy for RP.

To further investigate the epigenetic regulatory effects of EZH2 inhibition on the induction of anti-inflammatory responses, EZH2-related signaling pathways were also investigated. In protein level analysis, the expression levels of p-STAT1, STAT1, and IRF1 remained similar in the Gel only group and Control groups (compare the first two columns in FIG. 5D). That is, hydrogel treatment did not affect epigenetic regulatory signaling. In contrast, the Drug only group and Drug & Gel group down-regulated p-STAT1 and IRF1 protein expression, but maintained the total protein of STAT1, indicating successful inhibition of IRF1/STAT1 expression (FIG. 5D). These results show that the EZH2 inhibitor effect was maintained even when loaded on Hydrogel. The result showed that hydrogel with EZH2 inhibitors effectively weakened IRF1/STAT1 expression that regulates inflammatory mediums in inflammatory microglia. Overall, IRH loaded with EZH2 inhibitor showed anti-inflammatory potential in inflammatory microglia by reducing IRF1/STAT1 signaling that regulates inflammation (FIG. 5E).

2-3. Demonstration of the Protective Effect of IRH Loaded with EZH2 Inhibitor Against Photoreceptor Apoptosis

Prior to intraocular injection, the injectability of the hydrogel was tested using 31 G and 33 G Hamilton needles. The hydrogel was injectable at all crosslinker concentrations, such as 0.25, 0.5, and 1 mM. Since intraocular injection of IRH may increase intraocular pressure (IOP), thus the IOP in mouse eyes was measured both before and after injection. First, 1 μL of hydrogel prepared with 1 mM crosslinker was injected into wt and rd10 mice (see Experiment section). IOP was analyzed both after 3 hours of stabilization (in other words, day 0) and 2 weeks (in other words, day 14) following injection. Although the IOP measurements do not show transient changes, measurements suggest that IRH infusion did not alter IOP after recovery from infusion, and IOP remained within the normal IOP range (10-20 mmHg) in both wt and rd10 mice. These results confirm that the prepared IRH was unlikely to cause IOP-related adverse effects such as glaucoma.

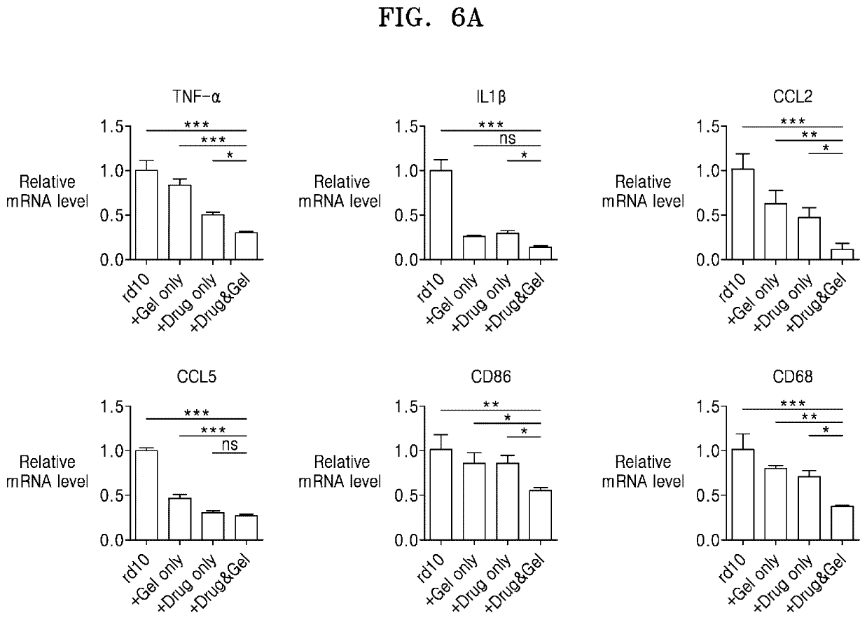

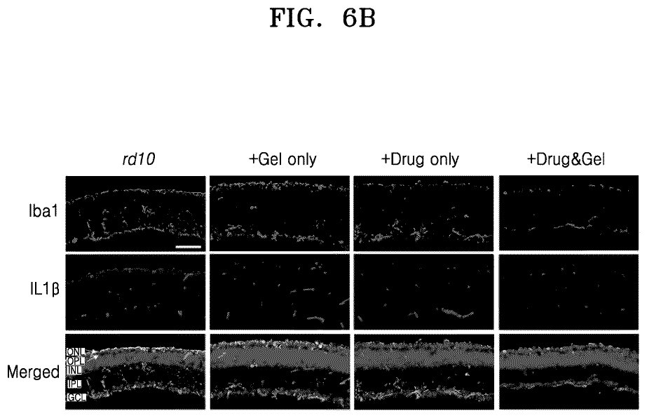

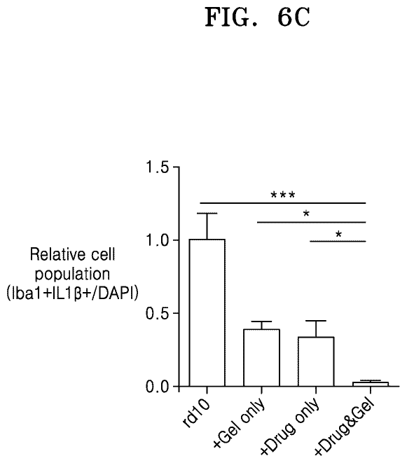

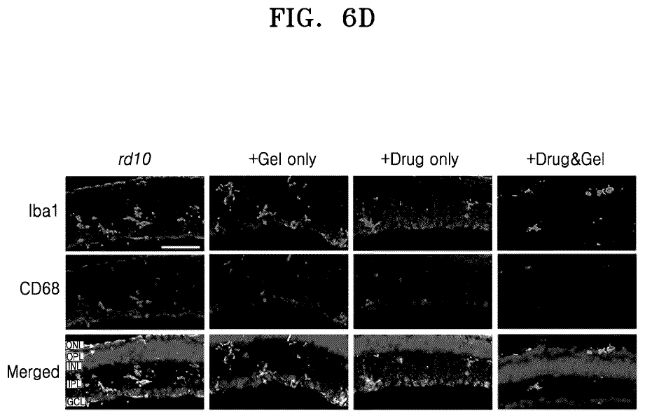

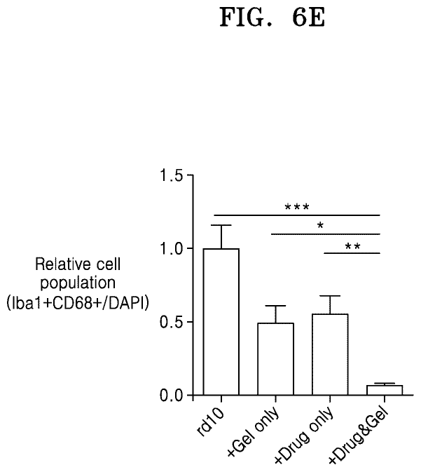

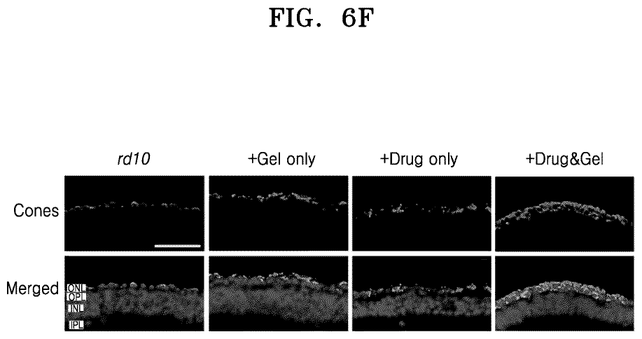

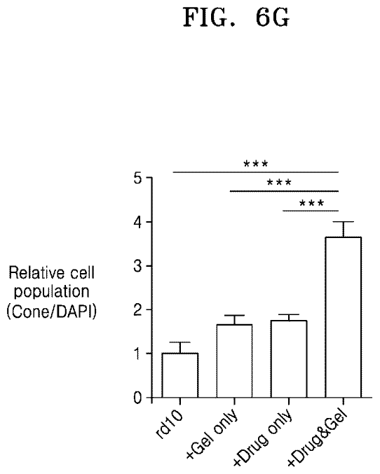

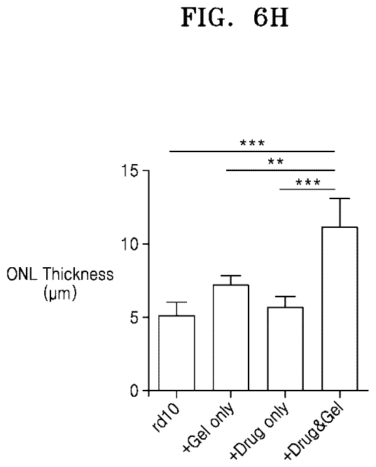

The therapeutic efficacy of IRH loaded with the EZH2 inhibitor was investigated in vivo using the widely studied RP model, rd10 mice. Hydrogels and/or drugs were injected intravitreally into three different groups of 5-week-old (in other words, postnatal week 5, or PW5) rd10 mice. 1) Hydrogel without drug (Gel only group; n=6), 2) EZH2 inhibitor in solution form without hydrogel (Drug only group; n=6), 3) Hydrogel loaded with EZH2 inhibitor (Drug & Gel group; n=6). Two weeks after injection (in other words, PW7), retinal tissue was harvested and analyzed at a genetic and protein level to investigate an extent of inflammatory activity. First, the mRNA levels of six inflammatory markers (in other words, TNF-α, IL1β, CCL2, CCL5, CD86, and CD68) were graphically represented for the three groups that received intravitreal injection (blue, red, and green bar in FIG. 6) and the control rd10 mice (no injection, black bar in FIG. 6). Consistent with the previous results (FIG. 5C), all markers were down-regulated in both the Drug only group and the Drug & Gel group compared to the rd10 group. This demonstrates the therapeutic effect of EZH2 inhibitors in an in vivo model (compare black and red/green bars in FIG. 6A). However, it is important to note that when EZH2 inhibitors were delivered together with IRH, the inflammatory response was reduced even more significantly (compare red and green bars in FIG. 6A). This demonstrates that the guided drug delivery capacity of the hydrogel was successful. On-demand drug release through hydrogels may lead to these results, which may maintain the therapeutic effect of the drug in the eye. Gel treatment alone induced some anti-inflammatory effects at an mRNA level (compare black and blue bars in FIG. 6A). These results demonstrate the anti-inflammatory effect of an HA hydrogel itself.

Additional immunofluorescence staining was also performed to localize inflammatory activity in the retinal tissue. Compared with the control group, microglia (Iba1) recruited in the retina was the lowest in the Drug & Gel group, but significantly decreased in both the Gel only group and the Drug only group (first row of FIG. 6B). A reduced number of recruited microglia indicates a reduced inflammatory response. In addition, the number of inflammatory microglia (stained with both Iba1 and IL1β) was effectively reduced in the Drug & Gel group (third row in FIG. 6B). Quantification of the number of stained microglia clearly showed a significant decrease in the Drug & Gel group (0.023±0.018; all data in FIG. 6C, FIG. 6E, and FIG. 6G are normalized to the control group in each group) (FIG. 6C). Microglia phagocytosis is known to occur in an inflammatory environment. Accordingly, in the retina tested, both Iba1 and CD68 were expressed to identify phagocytic microglia (FIG. 6D), clearly demonstrating a significant reduction in phagocytosis. A fraction of phagocytic microglia was most significantly reduced when the EZH2 inhibitor was delivered together with IRH (0.061±0.013) (FIG. 6E).

Finally, to determine whether the photoreceptors are preserved by the treatment, cone cells were stained using immunofluorescence (FIG. 6F). As seen in the much thicker and brighter green signal band in the upper right image, cone cells appeared to be most preserved in the Drug & Gel group (3.64±0.36). Additional quantitative counting results of cone cells showed that Drug & Gel treatment significantly improved cone cell viability by up to approximately 4 times compared to the control group (1.00±0.25) and approximately 2 times compared to the Gel only group (1.63±0.24) and Drug only group (1.75±0.15) (FIG. 6G). Additionally, the thickness of the outer nuclear layer (ONL; shown in green in the upper row of FIG. 6F), another indicator of degenerative progression in RP, was measured. In the untreated rd10 mouse group, ONL thickness decreased to 5.05±0.99 μm (black bar in FIG. 6H), but it was maintained at 11.15±1.96 μm in the Drug & Gel group (green bar in FIG. 6H). This was similar to an initial ONL thickness at the time of treatment (˜13.25 μm). All these results indicate that Drug & Gel treatment effectively delayed the pathological progression of retinal degenerative diseases and preserved cone cells. Overall, on-demand delivery of EZH2 inhibitors using IRH may effectively alleviate vision loss due to RD.

2-4. Demonstration of the Delaying Effect of IRH Loaded with EZH2 Inhibitor on Retinal Degeneration

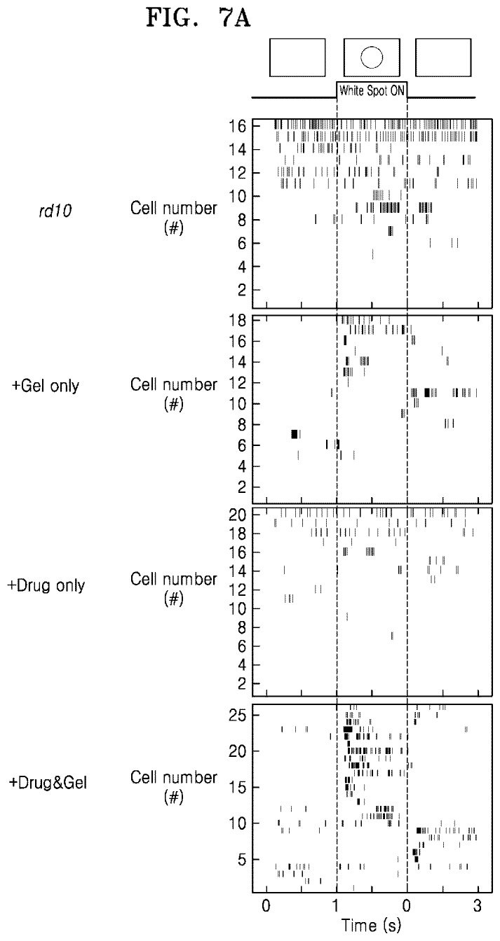

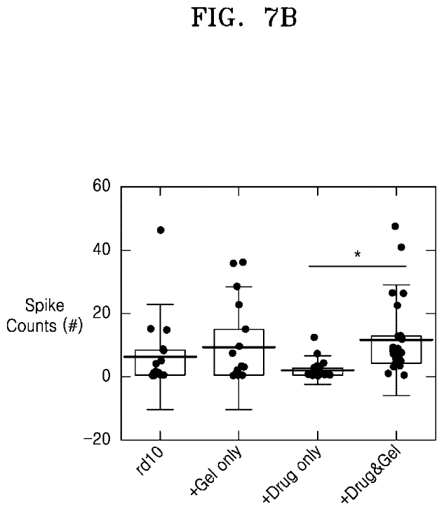

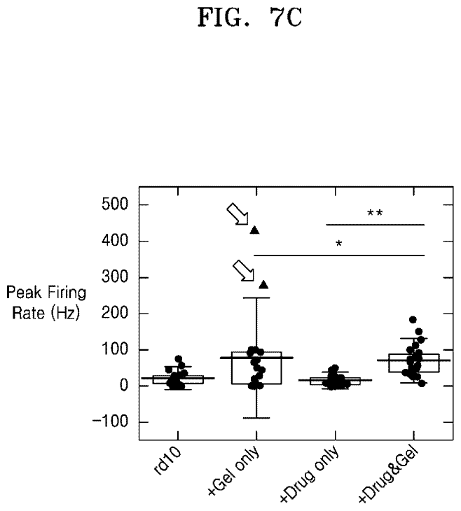

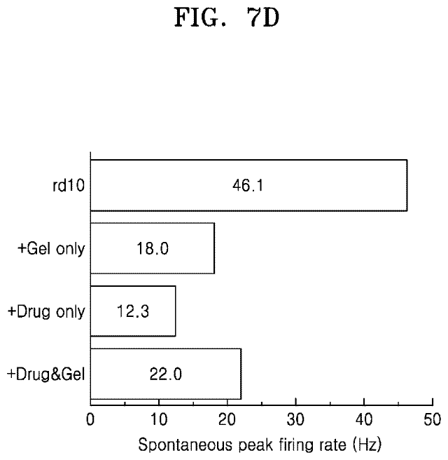

In the retina, rod cells and cone cells convert light signals into physiological signals, and then postsynaptic neurons, such as bipolar cells, horizontal cells, and amacrine cells, perform complex neural computations. In the final stage of the retina, retinal ganglion cells (RGCs) transmit visual information to the brain in the form of spike trains. Therefore, degeneration/apoptosis of photoreceptor cells significantly alters the spike activity of RGCs. To investigate a potential delay in physiological visual function loss after treatment with IRH, light-induced responses were recorded from RGCs in untreated rd10 mice and rd10 mice treated with Gel only, Drug only, and Drug & Gel (FIG. 7A).

The 1-sec-long white spot was projected onto the photoreceptor layer of target cells. Blue vertical band indicates the time range of spot flash was exposed. An induced spike represents an ON response. Conversely, a red vertical band represents an OFF response.

In response to stationary 1-sec-long white spot flashes, spiking responses were consistently elicited in RGCs of the Drug & Gel treated mice, and responses were strong enough to unambiguously classify most RGCs (24 cells out of 27 recorded cells) into either ON or OFF type (bottom raster plot in FIG. 7A). However, the rd10 and Drug only groups showed spontaneous spiking activity or showed multiple spikes transiently during the entire recording period (first and third raster plots in FIG. 7A). These results are representative of a phenomenon observed in degenerative retinas, which are known to be caused by fluctuations in the cell membrane potential of RGCs. In contrast to the rd10 and Drug only groups, RGC types (in other words, ON or OFF) could be distinguished in some cells in the Gel only group (second raster plot in FIG. 7A). However, as seen in the rd10 and Drug only groups, there are also RGCs that do not respond to light stimulus at all.

For more quantitative analysis, the number of spikes and peak firing rate (PFR) of the photoreaction were also calculated (FIG. 7B and FIG. 7C). RGCs in the Drug & Gel group generated an average of 11.57±11.59 spikes and showed an average PFR of 70.06±40.57 Hz. In contrast, the average spike numbers of the rd10, Gel only, and Drug only groups (6.30±11.07, 9.44±12.70, and 2.17±3.01 spikes, respectively) were relatively lower than the spike numbers of mice administered Drug & Gel (FIG. 7B). Although only one statistical significance was found in the spike count between the Drug only group and the Drug & Gel group (p<0.05), PFR analysis clearly showed statistical significance compared to the Drug & Gel group due to the increased spontaneous spike activity (FIG. 7D). The mean PFR of the rd10 (22.48±20.90 Hz; p<0.05) and Drug only (15.33±14.96 Hz; p<0.01) groups was significantly lower than that of the Drug & Gel group (70.06±40.57 Hz). However, in the case of the Gel only group, the mean PFR was higher and the standard deviation was also higher (77.35±110.18 Hz) than the other groups due to two outlier RGCs (indicated by blue arrows in FIG. 7C). In the absence of these two outliers, the PFR decreased to 42.83±39.54 Hz. It is particularly noteworthy that in spike activity analysis, the Drug only group did not show any stored physiological function of photoreceptors (compare red and green plots in FIG. 7B and FIG. 7C). This contrasts with the similar levels of anti-inflammatory effects between the Drug only group and the Drug & Gel group, which were confirmed by in vivo and in vitro mRNA and immunofluorescence analyses (FIG. 5 and FIG. 6). The enhanced ability of Drug & Gel treatment to protect photoreceptor function may be due to sustained drug release properties IRH (FIG. 4).

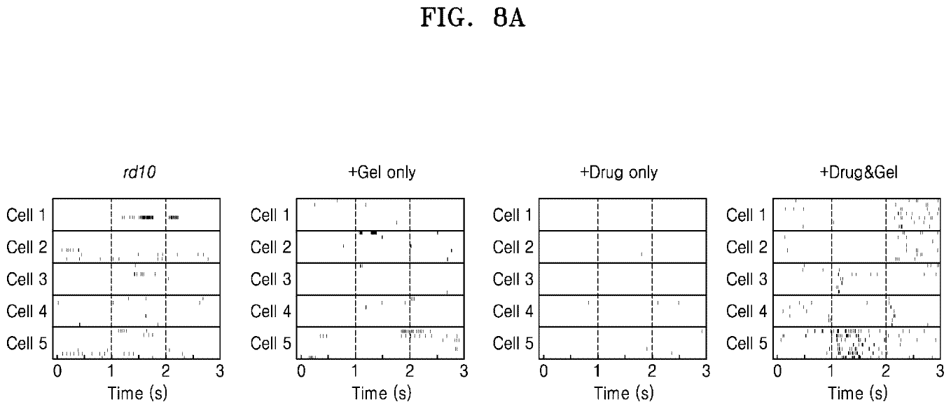

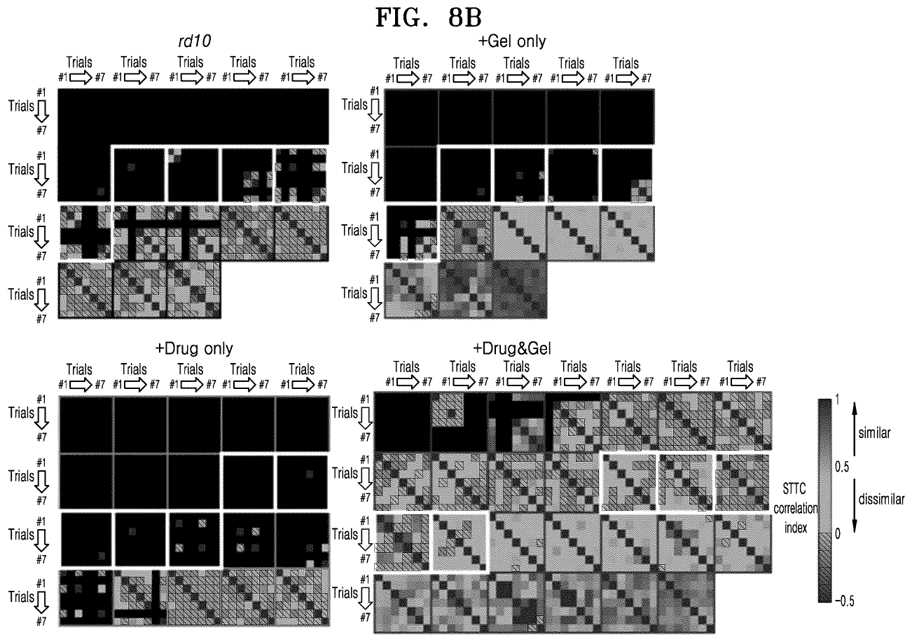

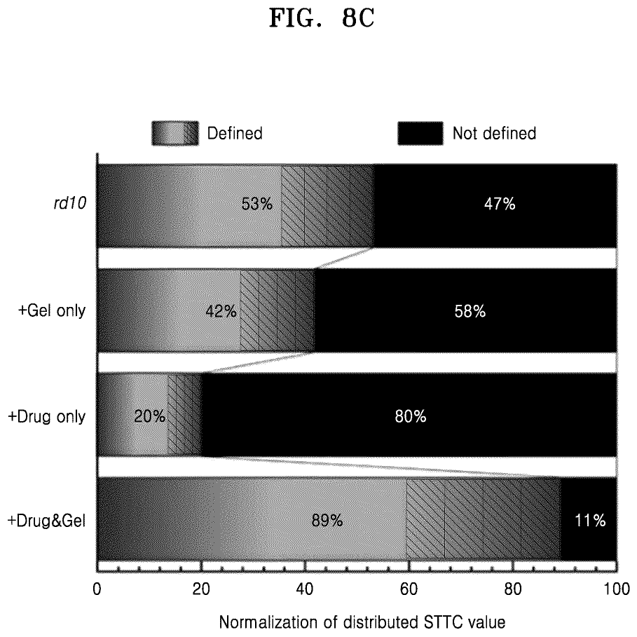

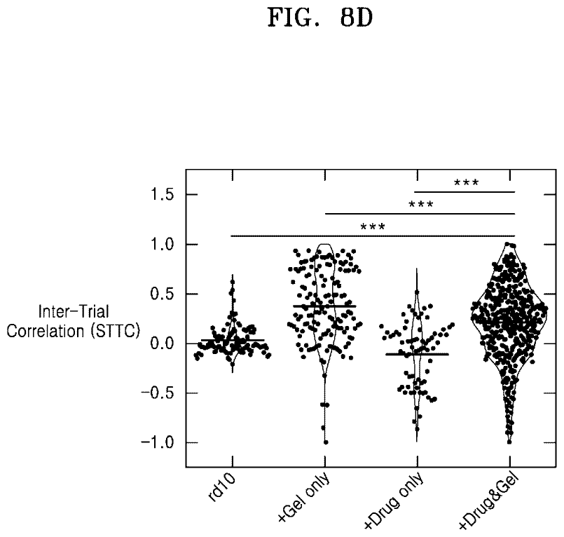

In a healthy retina, repeated repetitions of the same stimulus elicit coherent spike activity (in other words, similar spike trains occur at different repetitions), whereas in degenerated retinas, coherence is known to be reduced. To visualize the trial-to-trial consistency of light responses recorded from five representative RGCs, the spike activity induced by seven repetitions of the same light stimulus were shown in a graph (FIG. 8A). The rd10, Gel only, and Drug only groups showed weak and inconsistent spike patterns in response to the same light stimulus (FIG. 8A). Additionally, a spike time tiling coefficient (STTC) was calculated (see Equation 1 in the Experimental Methods section) at the corresponding repetition of each RGC and then a STTC matrix for all measured RGCs was shown in a graph (FIG. 8B). As shown in the first three subpanels of FIG. 8B, a black area of a STTC heat map, indicating an absence of spike activity, was larger in all groups except the Drug & Gel group. To quantify a fraction of non-responsive RGCs in each group, the percentage of defined and undefined STTC values was additionally calculated and then normalized to the number of recorded RGCs (FIG. 8C). Although STTC was still calculated in about half of the RGCs in rd10 mice (FIG. 8C, first row), this was mainly due to increased spontaneous activity (FIG. 7D): the peak spontaneous firing rates were 46.1, 18.0, 12.3, and 22.0 Hz in the rd10, Gel only, Drug only, and Drug & Gel groups, respectively. In particular, the higher the spontaneous activity, the closer the STTC was to 0 on average (black plot in FIG. 8D). In stark contrast, STTC was well defined in most RGCs in the Drug & Gel group (STTC values were defined in 89% of all recording pairs, as shown in the last row of FIG. 8C). The average STTC was also higher than that of the control group (0.22±0.37 and 0.03±0.14 in the case of the Drug & Gel group and the rd10 group, respectively; FIG. 8D).

Once again, it is very interesting that the therapeutic effect of the Drug only group performed worse in the response consistency analysis, which was worse than the results of the Gel only group (compare the middle two rows/columns in FIG. 8C and FIG. 8D). This trend was consistent with the response size analysis (FIG. 7B and FIG. 7C). These results may have two implications: 1) A single injection of EZH2 inhibitor may not be therapeutically effective, possibly due to rapid posterior clearance, and 2) long-term implantation of HA-based hydrogels may protect the physiological function of photoreceptors by reducing inflammatory activity (FIG. 6). In fact, the average STTC of the Gel only group was higher than that of the Drug & Gel group (0.38±0.40 in the case of the Gel only group and 0.22±0.37 in the case of the Drug & Gel group, FIG. 8D). However, this high result was due to two outlier cells mentioned in the PFR analysis (FIG. 7C). These showed a red hot STTC matrix (the last two in the second sub-panel of FIG. 8B) and the data points were clustered around 1 in the violin plot (blue graph in FIG. 8D). Except for these two RGCs, most RGCs in the Gel only group showed no light-induced response, and had the second highest percentage of undefined STTC values (in other words, 58% of all trials) (FIG. 8C).

In FIG. 8C, the percentage of STTC values defined in the control group (53%) was higher than those in the Gel only group (42%) and the Drug only group (20%). This was mainly because spontaneous activity was relatively higher in the rd10 group and the Drug & Gel group (FIG. 7D). However, as mentioned earlier, the calculated mean STTC of the rd10 group was low due to inconsistent responses. Notably, several RGCs in the Drug & Gel group also showed spontaneous activity throughout the pre-stimulus period (FIG. 7A), but light-induced responses were relatively strong and consistent after light stimulus.

Overall, the results suggest that treatment with IRH loaded with EZH2 inhibitors may effectively delay the deterioration of physiological function of the retina in addition to reducing the inflammatory activity that occurs in RP.

### Experimental Methods

### Mechanical Property Measurement

The flow properties of the prepared hydrogels were analyzed using a rheometer (Anton Paar, Graz, Austria). Measurements were performed at room temperature and at vibration frequencies ranging from about 0.1 to about 100 rad/sec at a 0.1% strain. Both a storage modulus (G′) and loss modulus (G″) were calculated by a rheometer software. Additionally, the stiffness of the hydrogel was analyzed using Instron (Instron 5900, Instron Corporation, Norwood, Massachusetts, USA). The hydrogel was compressed to measure the stress-strain curve, and Young's modulus was obtained from the slope of the linear region (the first 10% of strain) of the stress-strain curve.

### Scanning Electron Microscope (SEM) Imaging

The lyophilized hydrogel was placed in liquid nitrogen to obtain a clear cross-section of the sample during a cutting process. The frozen samples were cut with a blade. The cut samples were then placed on carbon tape, the surface was coated with platinum, and then loaded into a SEM (Teneo VS™, Thermo Fisher Scientific, Waltham, Massachusetts, USA) to image the prepared samples.

Characterization of Drug Release Profile from Inflammatory Responsive Hydrogel

Dextran (molecular weight: about 4 kDa) was loaded into a hydrogel and a dye release rate was quantified at various concentrations of cathepsin. Dextran-loaded hydrogels were placed into three 24-well transwell inserts and cultured in control medium (DMEM/F-12 medium), microglial conditioned medium, or activated microglia in DMEM/F-12 medium maintained at 37° C. Microglial conditioned medium was prepared by culturing microglia in DMEM/F-12 for 1 day. Conditioned medium for activated microglia was prepared by inducing inflammatory microglia with 50 ng/ml of TNF-α (#210-TA, R&D System) for 1 day in DMEM/F-12. Conditioned medium of activated microglia was harvested from the medium supernatant cultured in DMEM/F-12 for 1 day. Inserts were transferred to new 24-well plates, and fluorescence intensity was measured at 0, 1, 3, and 5 days using an in vivo imaging system (IVIS; Caliper Life Sciences, Waltham, Massachusetts, USA). In addition, hydrogels loaded with an EZH2 inhibitor Tazemetostat (EPZ-6438, Selleckchem, Houston, Texas, USA) were cultured in control medium (DMEM/F-12 medium), microglia-conditioned medium, and activated microglia in DMEM/F-12 medium maintained at 37° C. The supernatant was collected at 0.125, 0.25, 0.5, 1, 3, and 5 days and quantified by ultraviolet (UV) ray absorbance at 256 nm. Then, a release profile of the EZH2 inhibitor was investigated in response to various concentrations (in other words, 10 and 100 ng/ml) of cathepsins (L, S, and B subtypes all mixed) added to phosphate-buffered saline (PBS) at 37° C. For up to 5 days, the supernatant was collected daily and measured for UV absorbance. Cathepsin was added at all time points, 0.125, 0.25, 0.5, 1, 3 days.

### Cell Culture for In Vitro Experiments

Human microglial cell line SV40 was purchased from Applied Biological Material Inc. (T0251, Milton, Canada) and cultured in Dulbecco's Modified Eagle Medium/Nutrient Mixture F-12 (DMEM/F-12 in 1:1) (#11320-033, ThermoFisher, Waltham, Massachusetts, USA) supplemented with 10% fetal bovine serum (FBS) and 1% penicillin/streptomycin (PS). In the case of SV40 cell culture, collagen solution (50 μg/mL in 20 mM acetic acid) was coated on a culture plate and then washed three times with PBS before culturing SV40 cells. Mouse fibroblasts (NIH3T3) were obtained from the American Type Culture Collection (NIH/3T3-CRL-1658, Manassas, VA, USA) and cultured in DMEM (#11995-065, ThermoFisher, Waltham, Massachusetts, USA) supplemented with 10% FBS and 1% PS. Cells were cultured in a humidified atmosphere with 5% CO₂ at 37° C.

### In Vitro Cytotoxicity Assay of Hydrogel

The in vitro cytotoxicity of the prepared hydrogel was investigated using the Cell Counting Kit-8 assay (CCK-8, Dojindo, Kumamoto, Japan). NIH3T3 cells were seeded in a 24-well plate and cultured with hydrogels placed in 24 transwell inserts. Fibroblasts (NIH3T3) are a representative cell source for testing the cytotoxicity of biomaterials because these cells exhibit high growth rates and biological responses. In addition, it has been reported that retinal pigment epithelial-like cells and retinal ganglion-like cells may be derived from fibroblasts. Therefore, a cytotoxicity test of the hydrogel was performed using NIH3T3 cells. After culturing together with hydrogel, the cell was washed with PBS and soaked in a medium without DMEM including a 10% CCK-8 solution for 1.5 hours at 37° C. The supernatant was collected and measured for absorbance at 450 nm using a microplate reader. Live and dead cell staining was performed according to the manufacturer's protocol using Live & Dead/Cytotoxicity Assay Kit (#L3224, Invitrogen, Waltham, Massachusetts, USA). A mixture of 4 mM calcein AM (green) and 2 mM ethidium homodimer-1 (red) was added to the culture medium and cultured at 37° C. for 30 minutes. Stained cells were visualized using a confocal microscope (Carl Zeiss, Jena, Germany).

### In Vitro Study of Anti-Inflammatory Effects on Microglia

SV40 cells were cultured together with 50 ng/ml TNF-α (#210-TA, R&D systems, Minneapolis, Minnesota, USA) for 1 day to induce inflammatory microglia. To study the anti-inflammatory effect, inflammatory-activated microglia were treated under three different conditions: 1) hydrogel only (Gel only), 2) EZH2 inhibitor only (Drug only), and 3) hydrogel loaded with EZH2 inhibitor (Drug & Gel).

### Cathepsin Activity Assay

To confirm cathepsins secreted from inactivated microglia and activated microglia, conditioned media were harvested after culturing microglia in DMEM/F-12 medium for 1 day. The rd10 model retinal tissue was used for in vivo cathepsin activity assays. Fluorescence-based assay kits (#K142, #K144, #K140, Biovision, Milpitas, California, USA) were used to analyze the activity of cathepsins L, S, and B. The procedure was performed according to the manufacturer's instructions. The fluorescence intensity of the samples was measured using a fluorescence plate reader at 400/505 nm for excitation/emission wavelengths, respectively.

Quantitative Real-Time Polymerase Chain Reaction (Real-Time PCR) for mRNA Assay

Gene expression levels of cathepsins (cathepsins L, S, B) and inflammatory markers (in other words, TNF-α, IL1β, CCL2, CCL5, CD86, CD68) were analyzed by real-time PCR analysis. mRNA was isolated from microglia and retinal tissue using the RNeasy Mini Kit (#74104, QIAGEN, Hilden, Nordrhein-westfalen, Germany) and synthesized into cDNA using SuperScript IV VILO Master Mix (#11755050, Invitrogen, Waltham, Massachusetts, USA). Gene expression levels were performed using SYBR Premix Ex Taq (#RR420A, Takara, Kusatsu, Japan) according to the manufacturer's instructions. The sequences of all primers used in this study are presented in Table 1. Expressed gene levels were normalized to GAPDH, which serves as a housekeeping gene, and relative gene levels were calculated by the 2-ΔΔ^{CT} method.

TABLE 1

| | | Forward | | Reverse |
|---|---|---|---|---|
| Gene | No. | (5″→3″) | No. | (3″→5″) |
| hGAPDH | 2 | CACCATTGGCAA | 3 | AGGTCTTTGCGG |
| | | TGAGCGGTTC | | ATGTCCACGT |
| hTNF-α | 4 | GTGCCTATGTCT | 5 | GCCATAGAACTG |
| | | CAGCCTCTTC | | ATGAGAGGGA |
| hIL1β | 6 | CCACAGACCTTC | 7 | GTGCAGTTCAGT |
| | | CAGGAGAATG | | GATCGTACAGG |
| hCCL2 | 8 | AGAATCACCAGC | 9 | TCCTGAACCCAC |
| | | AGCAAGTGTCC | | TTCTGCTTGG |
| hCCL5 | 10 | CCTGCTGCTTTG | 11 | ACACACTTGGCG |
| | | CCTACATTGC | | GTTCTTTCGG |
| hCD86 | 12 | CCATCAGCTTGT | 13 | GCTGTAATCCAA |
| | | CTGTTTCATTCC | | GGAATGTGGTC |
| hCD68 | 14 | CGAGCATCATTC | 15 | ATGAGAGGCAGC |
| | | TTTCACCAGCT | | AAGATGGACC |
| hCathepsin | 16 | GAAAGGCTACGT | 17 | GTCTACCAGATT |
| L | | GACTCCTGTG | | CTGCTCACTC |
| hCathepsin | 18 | TGGATCACCACT | 19 | GCTCCAGGTTGT |
| S | | GGCATCTCTG | | GAAGCATCAC |
| hCathepsin | 20 | GCTTCGATGCAC | 21 | CATTGGTGTGGA |
| B | | GGGAACAATG | | TGCAGATCCG |
| mGAPDH | 22 | CATCACTGCCAC | 23 | ATGCCAGTGAGC |
| | | CCAGAAGACTG | | TTCCCGTTCAG |
| mTNF-α | 24 | GGTGCCTATGTC | 25 | GCCATAGAACTG |
| | | TCAGCCTCTT | | ATGAGAGGGAG |
| mIL1β | 26 | TGGACCTTCCAG | 27 | GTTCATCTCGGA |
| | | GATGAGGACA | | GCCTGTAGTG |
| mCCL2 | 28 | GCTACAAGAGGA | 29 | GTCTGGACCCAT |
| | | TCACCAGCAG | | TCCTTCTTGG |
| mCCL5 | 30 | CCTGCTGCTTTG | 31 | ACACACTTGGCG |
| | | CCTACCTCTC | | GTTCCTTCGA |
| mCD86 | 32 | ACGTATTGGAAG | 33 | TCTGTCAGCGTT |
| | | GAGATTACAGCT | | ACTATCCCGC |
| mCD68 | 34 | GGCGGTGGAATA | 35 | AGCAGGTCAAGG |
| | | CAATGTGTCC | | TGAACAGCTG |
| mCathepsin | 36 | GGAAAATGGAGG | 37 | GTGTCATTAGCC |
| L | | TCTGGACTCG | | ACAGCGAACTC |
| mCathepsin | 38 | GCATAGAGGCAG | 39 | CCACTGCTTCTT |
| S | | ACGCTTCCTA | | TCAGGGCATC |
| mCathepsin | 40 | AGTCAACGTGGA | 41 | GTAGACTCCACC |
| B | | GGTGTCTGCT | | TGAAACCAGG |

### Immunofluorescent Staining

For in vivo immunofluorescence staining, retinal tissues were fixed with 4% paraformaldehyde for 15 minutes and then included in optimal cutting temperature (OCT) compound and frozen. Tissues sectioned into 7 μm cryosections were probed with anti-Iba1 antibody (#sc-12742, Santa Cruz, Dallas, Texas, USA) at 1:250 dilution, anti-IL1β antibody (#ab183218, Abcam, Cambridge, Cambridgeshire, UK) at 1:200 dilution, anti-TNF-α antibody (#MCA1957T, Bio-Rad, Hercules, Contra Costa County, CA, USA) at 1:500 dilution, anti-CD68 antibody (#AB15282, Sigma-Aldrich, St. Louis, Missouri, USA) at 1:100 dilution, anti-Cone arrestin antibody (#ab133641, Abcam, Cambridge, Cambridgeshire, UK) at 1:500 dilution, anti-Cathepsin L antibody (#sc-271619, Santa Cruz, Dallas, Texas, USA) at 1:100 dilution, and primary antibodies were anti-Cathepsin S antibody (#ab214428, Abcam, Cambridge, Cambridgeshire, UK) at 1:300 dilution and anti-Cathepsin B antibody (H) at 1:250 dilution. Secondary antibodies were Donkey anti-rabbit IgG antibody (#A21207, Invitrogen, Waltham, Massachusetts, USA) diluted 1:500 and Goat anti-mouse IgG antibody (#A11001, Invitrogen, Waltham, Massachusetts, USA) diluted 1:500. Quantification of inflammatory microglia and cones was measured using immunofluorescence-stained retinal cryosections. Inflammatory microglia were counted in retinal sections using ImageJ, where yellow areas overlapped with green (Iba1) and red (IL1β or CD68). Cone cells were counted in the green area stained with cone arrestin antibody using ImageJ. The measured area was normalized by calculating the DAPI (blue) area of the retina. The thickness of the photoreceptor layer was averaged from randomly selected areas of three retinas to determine the precise effect of the hydrogel loaded with EZH2 inhibitor.

### Western Blot

Cell samples prepared for immunoblotting were lysed using RIPA lysis buffer (#R0278, Sigma-Aldrich, St. Louis, MO, USA) and protease and phosphatase inhibitor cocktail (#ab201119, Abcam, Cambridge, Cambridgeshire, UK). Equal amounts of protein were loaded onto 4% to 15% gradient gels (Bio-Rad Laboratories, CA, USA), transferred to polyvinylidene difluoride (PVDF) membranes (Bio-Rad Laboratories, CA, USA), and blocked by adding 5% BSA in TBST (TBS including 0.1% Tween 20) for 1 hour at room temperature. The membrane was cultured overnight at 4° C. in 5% BSA in TBST together with primary antibodies, including anti-GAPDH antibody (#ab8245, Abcam, Cambridge, Cambridgeshire, UK) at 1:5000 dilution, anti-Phospho-STAT (#9167, Cell Signaling, Danvers, MA, USA) at 1:1000 dilution, anti-STAT1 antibody (#9172, Cell Signaling) at 1:1000 dilution, and anti-IRF1 antibody (#8478, Cell Signaling) at 1:1000 dilution. The membrane was cultured with HRP-conjugated anti-mouse or anti-rabbit IgG (#7074, #7076, Cell Signaling) at a 1:1000 dilution for 1 hour at room temperature. Immunoblots were visualized with a chromilluminescence system (#34095, ThermoFisher, Waltham, Massachusetts, USA) and images were captured using an iBright CL1500 imaging system (ThermoFisher, Waltham, Massachusetts, USA).

### Animal Preparation

The animal study was approved by the Korea Institute of Science and Technology (KIST-5088-2022-05-077). All animal experiments were performed in accordance with institutional guidelines for the care and use of laboratory animals. Normal mice (C57BL/6J) were purchased from Daehan BioLink (Eumseong, South Korea). Retinal degeneration 10 (rd10) mice were used as a model of retinitis pigmentosa (RP). Breeding pairs of rd10 mice (B6.CXB1-Pde6b rd10/J) were purchased from the Jackson Laboratory (Bar Harbor, ME) and colonies were maintained in the KIST animal facility. The rd10 mouse is the most widely used animal model to study autosomal recessive RP models. Photoreceptor degeneration in rd10 mice is known to closely mimic the typical pattern of human RP, as light responses may be recorded for approximately one month after birth without overlapping with retinal development. However, rd10 mice may only express one RP genotype, which carries a missense mutation in the Pde6b gene (phosphodiesterase 6B, cGMP, rod receptor, beta polypeptide). Therefore, different rd mice are needed to include various RP genotypes, such as Crb1.

### Intravitreal Injection In Vivo and Retinal Tissue Preparation

To compare the anti-inflammatory effects of the materials, four different groups of mice were prepared: 1) uninjected rd10 control group, 2) hydrogel only (Gel only), 3) EZH2 inhibitor only (Drug only), and 4) hydrogel loaded with EZH2 inhibitor (Drug & Gel). During intravitreal injections, mice were maintained under anesthesia with isoflurane in oxygen. A small hole was made slightly posterior to the margin with a 30 G 1/2 needle to inject the prepared material. Then, 1-2 μL of prepared hydrogel, EZH2 inhibitor (Tazemetostat), was injected intravitreally into 5-week-old mice using a 33 G Hamilton needle (#NANOFIL, World Precision Instruments, Sarasota, Florida, USA). Two weeks after injection, the mice were anesthetized by inhaling evaporating isoflurane and sacrificed via cervical dislocation. The retinal tissue was then separated from the eyeball using fine forceps and used in bio experiments.

### Patch-Clamp Recording and Light Stimuli

Patch-clamping records were used to investigate a physiological function of retinal tissue at a single spike level of each individual RGC. Isolated retinas were mounted ganglion cell-side up on filter paper with a small hole (˜2 mm diameter) in the center to allow light stimuli. The prepared sample was placed on a slide glass and continuously perfused at a rate of 4 mL/min with oxygenated Ames' medium (Sigma Aldrich, St. Louis, MO, USA) while maintaining the temperature at 34° C. to 36° C. Spiking activity of RGCs was recorded using a cell-attached patch clamping method with glass pipettes (8 MΩ to 11 MQ). After removing the internal limiting membrane, alpha RGCs with large body diameter (>20 μm) were targeted. Two silver chloride-coated silver wires in the shape of a ball were placed on opposite sides of the recording chamber as ground electrodes. Data were low-pass filtered at 2 kHz using an amplifier (MultiClamp 700B, Molecular Devices, Sunnyvale, CA).

For light stimuli, stationary 1-sec-long white spot flashes on a gray background were focused at the photoreceptor layer and were delivered at various diameters ranging from 100 to 1000 μm. All visual stimuli were repeated 5 to 7 times. The recorded RGC numbers were 16, 21, 20, and 27 in the Control, Gel only, Drug only, and Drug & Gel groups, respectively.

### Analysis of Light-Evoked Spiking Responses

Patch-clamp recordings enabled sophisticated spike-level analysis of retinal neurons protected from degeneration. First, the timing of induced spikes was detected from raw recordings via a custom script written in MATLAB (MathWorks, Natick, MA, USA). Based on the light-induced response, RGCs were classified into ON, OFF, ON-OFF, and unknown (including no responsive cells or remaining cells other than the classified cells) subtypes. In the case of a direction-selective subtype, RGCs were not tested with moving stimuli. The spikes of ON and OFF RGCs were counted for 1-sec-long presentation of light stimulus (see red band in FIG. 7A) and for 1 sec from the offset of the light (see blue band in FIG. 7A), respectively. However, in case of the unknown and ON-OFF type of RGCs, because it was difficult to determine the response polarity (in other words, ON or OFF), the average value of spike count during the ON and OFF response periods (for 2 seconds; red and blue bands in FIG. 7A) was calculated. The highest number of spikes occurring at various spot sizes (in other words, 100 μm to 1000 μm) was selected for subsequent analysis. Firing rates were calculated using bin sizes of 20 ms shifted at 5 ms intervals. The peak firing rate (PFR) was the maximum value of the calculated firing rates. Additionally, the spike timing consistency was examined by calculating the Spike Time Tiling Coefficient (STTC) in the repetitions using the following Equation 1.

$$STTC = 1 / 2 \times [ ( {P}_{A} - {T}_{B} ) / ( 1 - {P}_{A} {T}_{B} ) + ( {P}_{B} - {T}_{A} ) / ( 1 - {P}_{B} {T}_{A} ) ] . \qquad [ Equation 1 ]$$

P^{A} was the proportion of spikes in A that fall within a ±time window (±Δt) of all spikes in B (P_{B} is calculated similarly). T_{A} was the proportion of total recording time that is within ±Δt of all spikes in A (T_{B} is calculated similarly). A Δt of 10 ms was used in this work.

The computed STTC was displayed as a color-coded heat map (FIG. 8B). Each column matrix of STTC computed in a given cell was surrounded by a thin colored border representing different groups (in other words, black, blue, red, and green for Control, Gel only, Drug only, and Drug & Gel, respectively). Black was used to indicate cases where STTC values are not defined due to lack of spike activity. The heat matrices were arranged in the following order: 1) Number of defined STTC values from low to high, 2) Average STTC value from low to high. Five RGCs from the center of the arranged column matrix were selected (see the thick white border surrounding them) and marked their raster plots as representative cells (FIG. 8A). The percentage of ‘undefined and ‘defined STTC values in each group was calculated (FIG. 8C). The defined STTC values were also displayed as data points in the violin plot (FIG. 8D). Spontaneous activity was evaluated by calculating the pre-stimulus period of 500 ms (FIG. 7D).

### Statistical Analysis

All data were expressed as mean±standard deviation. Data shown in FIG. 1, FIG. 4, FIG. 5, and FIG. 6 were analyzed using one-way analysis of variance (ANOVA) in Prism followed by Tukey's post hoc test. Data shown in FIG. 7 and FIG. 8 were analyzed using one-way analysis of variance (ANOVA) in Origin followed by the Holm-Sidak post hoc test. Statistical significance was *p<0.05, *p<0.01, *p<0.001, and *p<0.0001.

### CONCLUSION

In this study, an HA-based inflammatory responsive hydrogel was developed for on-demand anti-inflammatory therapy for retinal degenerative diseases. The inflammatory responsive hydrogel demonstrated controlled drug release in response to various concentrations of cathepsin and inflammatory conditions. The in vitro experiment showed that the hydrogel is suitable for intraocular injection due to its properties such as injectability, biocompatibility, and transparency. Furthermore, an on-demand anti-inflammatory effect on inflammatory microglia was achieved through the epigenetic regulation of the EZH2 inhibitor released from the hydrogel. Intravitreal injection of hydrogel loaded with EZH2 inhibitor into the outer retinal degeneration model rd10 mouse induced anti-inflammatory effects in the retinal environment and protected against cone cell apoptosis of photoreceptors. In particular, ganglion cell response results showed delayed retinal degeneration and vision loss in the rd10 mouse model. Overall, HA-based inflammatory responsive hydrogels showed efficient drug delivery depending on disease activity for anti-inflammatory treatment. This therapeutic strategy suggests potential for effective treatment in progressive retinal degenerative diseases. The hydrogel platform developed in this study is expected to be applicable to various inflammatory diseases that require minimally invasive treatment together with various drugs.

According to the hydrogel according to an aspect, the release of the drug may be controlled selectively for the inflammatory condition. Specifically, the cathepsin-cleavable peptide crosslinker included in the hydrogel may be degraded or cleaved by activated cathepsin, a biomarker of inflammatory response, thereby releasing the drug encapsulated within the hydrogel. Due to this, the hydrogel according to an aspect, may release the drug selectively only when exposed to an inflammatory environment, while delaying the release of the drug and protecting the drug from the external environment in a normal environment or an environment with a low concentration of activated cathepsin, and in particular, may continuously release the drug from the stage with a high concentration of activated cathepsin, in other words, the acute inflammatory phase, through the subsequent proliferative phase. As a result, the hydrogel according to an aspect, may control the release of the drug with maintained activity at the most appropriate time to increase the therapeutic efficiency throughout the entire course of the inflammatory response, thereby maximizing the effect of the drug and exhibiting remarkably excellent anti-inflammatory, cell regeneration, and wound healing effects. Furthermore, the hydrogel according to an aspect, may be directly injected into the eye.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A hydrogel formed by cross-linking hyaluronic acid with a crosslinker,
wherein a drug is encapsulated inside the hydrogel,
and the crosslinker comprises a peptide capable of being degraded by cathepsins.

2. The hydrogel of claim 1, wherein the hyaluronic acid is a dibenzylcyclooctyne (DBCO)-conjugated hyaluronic acid (DBCO-HA).

3. The hydrogel of claim 1, wherein the peptide capable of being degraded by cathepsins comprises an amino acid sequence of SEQ ID NO: 1.

4. The hydrogel of claim 1, wherein when the crosslinker is degraded by cathepsins, the drug is released to outside the hydrogel.

5. The hydrogel of claim 1, wherein the hydrogel selectively releases the drug at an inflammatory lesion.

6. The hydrogel of claim 1, wherein the drug is an inhibitor of enhancer of zeste homolog 2 (EZH2).

7. The hydrogel of claim 1, wherein the hydrogel is for ocular injection.

8. A method of ocular disease treatment, comprising administering a therapeutically effective amount of a hydrogel to a subject, wherein the hydrogel is a hydrogel formed by cross-linking hyaluronic acid with a crosslinker, wherein a drug is encapsulated inside the hydrogel, and wherein the crosslinker comprises a peptide that is degradable by cathepsins.

9. The method of claim 8, wherein the ocular disease is a degenerative retinal disease.

10. A method for preparing a hydrogel, comprising:
(1) preparing dibenzylcyclooctyne (DBCO)-conjugated hyaluronic acid (DBCO-HA);
(2) mixing the DBCO-HA with a solvent to prepare a mixture;
(3) mixing a drug into the mixture; and
(4) adding a crosslinker thereto,
wherein the crosslinker comprises a peptide that is degradable by cathepsins.

11. The method of claim 10, wherein the peptide degradable by cathepsins comprises an amino acid sequence of SEQ ID NO: 1.

12. The method of claim 10, wherein the drug is an inhibitor of enhancer of zeste homolog 2 (EZH2).
